(19) United States
(12) Patent Application Publication
(10) Pub. No.: US 2010/0249165 A1
(43) Pub. Date: Sep. 30, 2010

(54) PDZ DOMAIN MODULATORS

(75) Inventors: Ulrik Gether, Charlottenlund (DK); Kenneth Madsen, Virum (DK); Thor Seneca Thorsen, Copenhagen (DK); Dan Peters, Malmo (SE); Tino Dyhring, Solrod (DK); Lars Christian Rønn, Hillerod (DK)

Correspondence Address: BIRCH STEWART KOLASCH & BIRCH, PO BOX 747, FALLS CHURCH, VA, 22040-0747, US

(73) Assignees: University of Copenhagen, Kobenhavn N (DK); NeuroSearch A/S, Ballerup (DK)

(21) Appl. No.: 12/663,508

(22) PCT Filed: Jun. 3, 2008
(86) PCT No.: PCT/EP08/56814

§ 371 Date: Jun. 15, 2010

Related U.S. Application Data
(60) Provisional application No. 60/942,860, filed on Jun. 8, 2007.

(30) Foreign Application Priority Data
Jun. 8, 2007 (DK) PA 2007 00828

(51) Int. Cl. A61K 31/4418; A61P 25/06; A61P 25/16; A61P 25/18; A61P 25/22; A61P 25/24; A61P 25/28; A61P 9/04; A61P 9/10; A61P 25/08; A61P 13/06; A61P 1/08; A61P 3/04; A61P 25/30; A61P 25/36; A61K 31/11; C07C 47/575; C07D 213/48; C07D 307/46; A61K 31/341; C07D 333/22; A61K 31/381; C07C 255/44; A61K 31/277; C07D 333/24; A61K 31/352; C07D 311/14; C07D 409/12

(52) U.S. Cl. 514/277; 514/699; 568/442; 546/340; 549/498; 514/461; 549/78; 514/438; 558/401; 514/482; 549/496; 514/471; 514/457; 549/399; 549/57; 514/443

(57) ABSTRACT

This invention relates to compounds useful as PDZ domain modulators, in particular the PDZ domain of PICK1.

In other aspects the invention relates to the use of these compounds in a method for therapy and to pharmaceutical compositions.

### TECHNICAL FIELD

This invention relates to compounds useful as PDZ domain modulators, in particular the PDZ domain of PICK1.

In other aspects the invention relates to the use of these compounds in a method for therapy and to pharmaceutical compositions.

### BACKGROUND ART

PDZ (PSD-95/Discs-large/ZO-1 homology) domains are one of the most common protein domains in the human genome with over 540 domains in more than 300 different proteins. They mediate cellular protein-protein interactions and serve important roles in protein targeting and in the assembly of protein complexes. PICK1 (Protein interacting with C Kinase 1) contains a single N-terminal PDZ domain and was originally identified as a protein interacting with protein kinase Cα (PKCα). In addition to its N-terminal PDZ domain, PICK1 contains a coiled-coil domain (residue 145-165), which is believed to mediate dimerization of PICK1. This is followed by a region with homology to Arfaptin 1 and 2 (residue 152-362), and a C-terminal acidic cluster (residue 381-389).

Functionally, PICK1 protein has been shown to be important for regulation of signaling through the AMPA receptor. PICK1 interacts with the AMPA receptor via binding of the C-terminal 3-4 residues of the GluR2 subunit in its single N-terminal PDZ domain. This interaction has, depending on cell type, been shown to be a positive and a negative regulator of the levels of GluR2 at the plasma membrane, thus affecting the molecular composition and gating properties of the AMPA receptor. Most importantly a recent study has shown that disruption of the PICK1 interaction with the GluR2 subunit by intrathecal injection of membrane permeable peptides that specifically bound to the PDZ domain of PICK1 showed efficacy in an animal model for neuropathic pain. These data suggest that the PDZ domain of PICK1 might in particular be a relevant target for treatment of pain, such as neuropathic pain.

### SUMMARY OF THE INVENTION

It is the object of the invention to provide small molecule inhibitors that target the PDZ domain, and in particular the PDZ domain of PICK1, preferably compounds which bind with high affinity and high specificity to the PDZ domain of PICK1.

In its first aspect, the invention provides the use of a compound of Formula 1a, 1b, 1c, 1d, 1e or 1f:

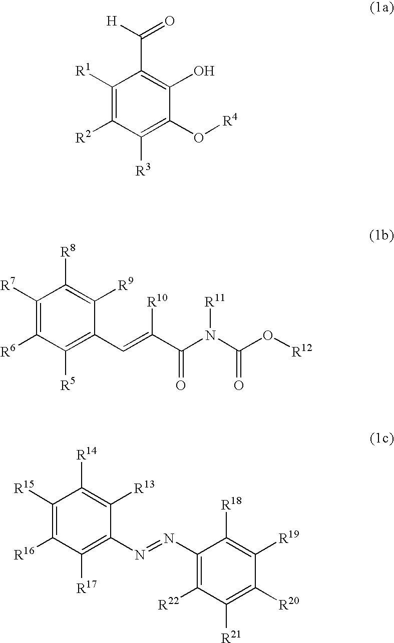

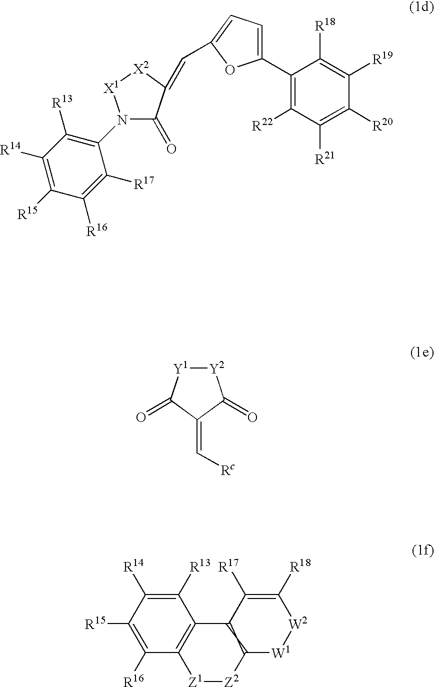

any of its stereoisomers or any mixture of its stereoisomers, or a pharmaceutically acceptable salt thereof; for the manufacture of a pharmaceutical composition for the treatment, prevention or alleviation of a disease or a disorder or a condition of a mammal, including a human, which disease or disorder or condition is responsive to modulation of a PDZ domain; where in Formula 1a, 1b, 1c, 1d, 1e and 1f, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R^{c}, X¹, X², Y¹, Y², Z¹, Z², W¹ and W² are as defined below.

In a further aspect, the invention relates to a method for treatment, prevention or alleviation of a disease or a disorder or a condition of a living animal body, including a human, which disorder, disease or condition is responsive to modulation of a PDZ domain, which method comprises the step of administering to such a living animal body in need thereof a therapeutically effective amount of a compound for use according to the invention, any of its stereoisomers or any mixture of its stereoisomers, or a pharmaceutically acceptable salt thereof.

In a still further aspect, the invention relates to novel compounds of Formula Ia and Ib, any of its stereoisomers or any mixture of its stereoisomers, or a pharmaceutically acceptable salt thereof.

Other objects of the invention will be apparent to the person skilled in the art from the following detailed description and examples.

### DETAILED DISCLOSURE OF THE INVENTION

In its first aspect the present invention provides the use of a compound of Formula 1a, 1b, 1c, 1d, 1e or 1f:

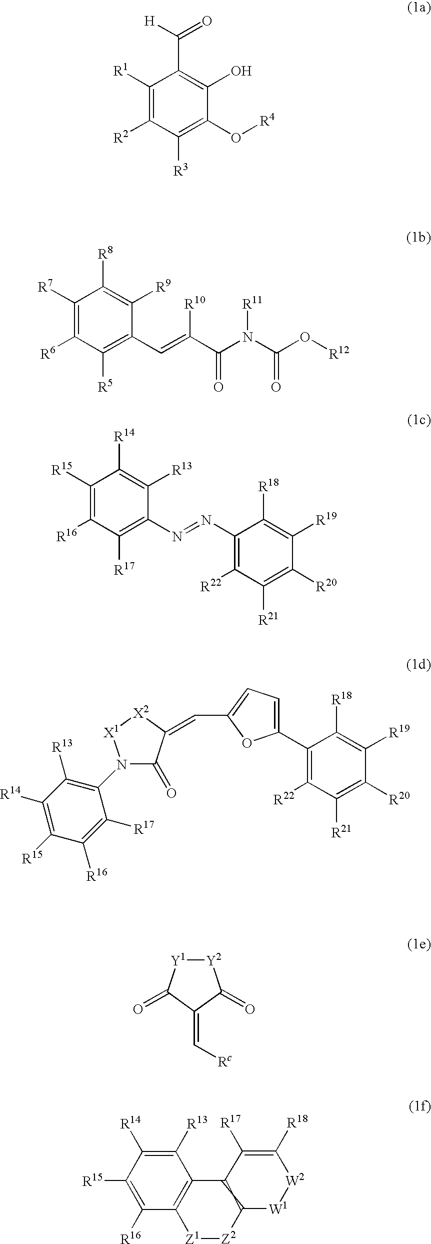

any of its stereoisomers or any mixture of its stereoisomers,
or a pharmaceutically acceptable salt thereof;
for the manufacture of a pharmaceutical composition for the treatment, prevention or alleviation of a disease or a disorder or a condition of a mammal, including a human, which disease or disorder or condition is responsive to modulation of a PDZ domain;
where in Formula 1a, 1b, 1c, 1d, 1e and 1f,
R¹, R² and R³ are independently selected from the group consisting of:
hydrogen, halo, trifluoromethyl, trifluoromethoxy, cyano, nitro, alkyl, hydroxy, alkoxy, formyl, alkylcarbonyl and —(C≡C)ₙ—R^{a}; wherein
   n is 0 or 1; and
   R^{a} represents an aryl or a heteroaryl group;
      which aryl or heteroaryl group is optionally substituted with one or more substituents independently selected from the group consisting of:
         halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
         wherein R′ and R″ independent of each other are hydrogen or alkyl;
            R⁴ represents hydrogen or alkyl;
            R⁵, R⁶, R⁷ and R⁸ are independently selected from the group consisting of:
hydrogen, halo, trifluoromethyl, trifluoromethoxy, cyano, nitro, alkyl, hydroxy, alkoxy, formyl, alkylcarbonyl, or an aryl or a heteroaryl group;
   which aryl or heteroaryl group is optionally substituted with one or more substituents independently selected from the group consisting of:
      halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR(C═O)R″;
         wherein R′ and R″ independent of each other are hydrogen or alkyl;
            R⁹ and R¹⁰ together form —(O—(C═O))—, —O— or —S—; or
            R⁹ represents hydrogen or alkyl; and R¹⁰ represents hydrogen, cyano or alkyl;
            R¹¹ and R¹² together form —(CHR′—CH₂)—;

wherein R′ represents hydrogen, alkyl or phenyl; or

R¹¹ represents hydrogen or alkyl; and
R¹² represents hydrogen, alkyl, alkenyl or alkynyl;
which alkyl, alkenyl or alkynyl is optionally substituted with an aryl or heteroaryl group;
   which aryl or heteroaryl group is optionally substituted with one or more substituents independently selected from the group consisting of:
      halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
         wherein R′ and R″ independent of each other are hydrogen or alkyl
            R¹³, R¹⁴, R¹⁵, R¹⁶, R¹⁷, R¹⁸, R¹⁹, R²⁰, R²¹ and R²² are independently selected from the group consisting of:
hydrogen, halo, trifluoromethyl, trifluoromethoxy, cyano, nitro, alkyl, hydroxy, alkoxy, formyl, alkylcarbonyl, hydroxycarbonyl, alkoxycarbonyl, R^{b} and —C═N—R^{b};
wherein
   R^{b} represents an aryl or a heteroaryl group;
      which aryl or heteroaryl group is optionally substituted with one or more substituents independently selected from the group consisting of:
         halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
         wherein R′ and R″ independent of each other are hydrogen or alkyl;
            -X¹-X²- represents —N═(C—R′)— or —NR″—(C═O)—; wherein
wherein R′ and R″ independent of each other are hydrogen or alkyl;
   —Y¹—Y²— represents

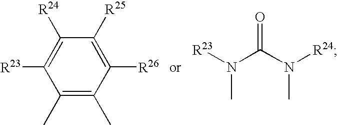

wherein
R²³, R²⁴, R²⁵ and R²⁶ are independently selected from the group consisting of:
   hydrogen, halo, trifluoromethyl, trifluoromethoxy, cyano, nitro, alkyl, hydroxy, alkoxy, formyl, alkylcarbonyl, and R^{d}; wherein
      R^{c} and R^{d} independent of each other represents an aryl or a heteroaryl group;
which aryl or heteroaryl group is optionally substituted with one or more substituents independently selected from the group consisting of:
   halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, R^{e}-alkoxy, —NR′R″, —(C═O)NR′R″ or —NR′(C═O)R″;
      wherein R′ and R″ independent of each other are hydrogen or alkyl;
      R^{e} represents an aryl group;
         which aryl group is optionally substituted with one or more substituents independently selected from the group consisting of:
         halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′″R″″, —(C═O)NR′″R″″ or —NR′″(C═O)R″″;
         wherein R′″ and R″″ independent of each other are hydrogen or alkyl;
            -Z¹-Z²- represents —NR′—C(COOR″)— or —(C═O)—(C═O)—; wherein
wherein R′ and R″ independent of each other are hydrogen or alkyl;
   —W¹—W²— represents —C(R²⁷R²⁸)— or —CR²⁷=CR²⁸—; wherein
wherein R²⁷ and R²⁸ are independently selected from the group consisting of:
hydrogen, halo, trifluoromethyl, trifluoromethoxy, cyano, nitro, alkyl, hydroxy alkoxy, formyl and alkylcarbonyl;
   the bond [symbol] represents a single or a double bond.

In one embodiment, the compound for use is a compound of Formula 1a

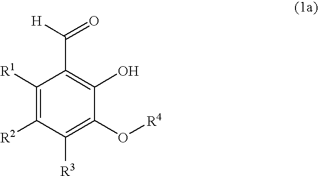

any of its stereoisomers or any mixture of its stereoisomers, or a pharmaceutically acceptable salt thereof; wherein R¹, R², R³ and R⁴ are as defined above.

In a special embodiment of the compound of Formula 1a, R¹, R² and R³ independent of each other represent hydrogen, halo or nitro. In a further embodiment, R¹ represents hydrogen. In a still further embodiment, R¹ represents halo, such as bromo or chloro. In a further embodiment, R² represents hydrogen. In a still further embodiment, R² represents halo, such as bromo or chloro. In a further embodiment, R² represents nitro. In a still further embodiment, R³ represents hydrogen.

In a further special embodiment of the compound of Formula 1a, R⁴ represents alkyl, such as ethyl.

In a further embodiment, the compound for use is a compound of Formula 1b

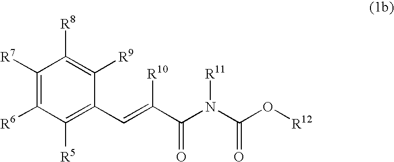

any of its stereoisomers or any mixture of its stereoisomers, or a pharmaceutically acceptable salt thereof; wherein R⁵, R⁶, R⁷, R⁸, R⁹, R¹⁰, R¹¹ and R¹² are as defined above.

In a special embodiment of the compound of Formula 1b, R⁵, R⁶, R⁷ and R⁸ independent of each other represent hydrogen or halo. In a further embodiment, R⁵ represents hydrogen. In a further embodiment, R⁶ represents hydrogen. In a further embodiment, R⁷ represents hydrogen. In a further embodiment, R⁷ represents halo, such as chloro. In a further embodiment, R⁸ represents hydrogen. In a further embodiment, R⁸ represents halo, such as chloro.

In a further special embodiment of the compound of Formula 1b, R⁹ and R¹⁰ together form —(O—(C═O))—. In a further embodiment, R⁹ and R¹⁰ together form —O—. In a still further embodiment, R⁹ and R¹⁰ together form —S—.

In a still further special embodiment of the compound of Formula 1b, R⁹ represents hydrogen or alkyl; and R¹⁰ represents hydrogen, cyano or alkyl. In a further embodiment, R⁹ represents hydrogen and R¹⁰ represents hydrogen. In a still further embodiment, R⁹ represents hydrogen and R¹⁰ represents cyano.

In a further special embodiment of the compound of Formula 1b, R¹¹ and R¹² together form —(CHR′—CH₂)—, wherein R′ represents hydrogen or phenyl. In a further embodiment, R′ represents hydrogen. In a still further embodiment, R′ represents phenyl.

In a further special embodiment of the compound of Formula 1b, R¹¹ represents hydrogen and R¹² represents optionally substituted alkyl, such as ethyl or n-butyl.

In a still further embodiment, the compound for use is a compound of Formula 1c

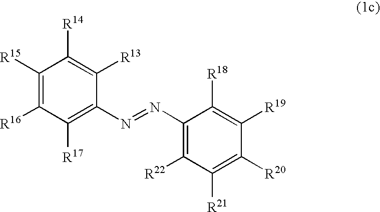

any of its stereoisomers or any mixture of its stereoisomers, or a pharmaceutically acceptable salt thereof; wherein R¹³, R¹⁴, R¹⁵, R¹⁶, R¹⁷, R¹⁸, R¹⁹, R²⁰, R²¹ and R²² are as defined above.

In a special embodiment of the compound of Formula 1c, four of R¹⁶, R¹⁷, R¹⁸, R¹⁹, R²⁰, R²¹ and R²² represent hydrogen. In a further embodiment, R²⁰ represents halo, such as chloro.

In a further special embodiment of the compound of Formula 1c, two of R¹⁶, R¹⁷, R¹⁸, R¹⁹, R²⁰, R²¹ and R²² represent hydrogen. In a further embodiment, three of R¹⁶, R¹⁷, R¹⁸, R¹⁹, R²⁰, R²¹ and R²², such as R¹⁹, R²⁰ and R²¹, independent of each other represent hydroxy, alkoxy, or formyl. In a still further embodiment, R¹⁹ represents alkoxy, such methoxy. In a further embodiment, R²⁰ represents hydroxy. In a still further embodiment, R²¹ represents formyl.

In a still further special embodiment of the compound of Formula 1c, four of R¹³, R¹⁴, R¹⁵, R¹⁶ and R¹⁷ represent hydrogen. In a further embodiment, one of R¹³, R¹⁴, R¹⁵, R¹⁶ and R¹⁷, such as R¹⁵, represents halo, such as bromo.

In a further special embodiment of the compound of Formula 1c, three of R¹³, R¹⁴, R¹⁵, R¹⁶ and R¹⁷ represent hydrogen. In a further embodiment, two of R¹³, R¹⁴, R¹⁵, R¹⁶ and R¹⁷, such as R¹⁴ and R¹⁵, independent of each other represent hydroxy or —C═N—R^{b}; wherein R^{b} represents optionally substituted phenyl, such as alkyl-hydroxy-phenyl, such as 5-tertbutyl-2-hydroxy-phenyl. In a still further embodiment, R¹⁴ represents —C═N—R^{b} and R¹⁵ represents hydroxy.

In a further embodiment, the compound for use is a compound of Formula 1d

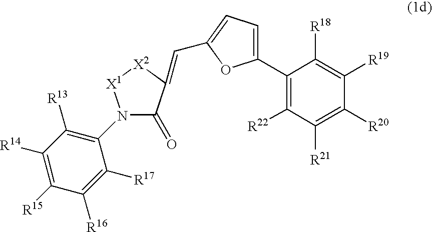

any of its stereoisomers or any mixture of its stereoisomers, or a pharmaceutically acceptable salt thereof; wherein R¹³, R¹⁴, R¹⁵, R¹⁶, R¹⁷, R¹⁸, R¹⁹, R²⁰, R²¹, R²² and -X¹-X²- are as defined above.

In a special embodiment of the compound of Formula 1d, four of R¹³, R¹⁴, R¹⁵, R¹⁶ and R¹⁷ represent hydrogen. In a further embodiment, one of R¹³, R¹⁴, R¹⁵, R¹⁶ and R¹⁷, such as R¹⁴, represents hydroxycarbonyl.

In a further special embodiment of the compound of Formula 1d, three of R¹³, R¹⁴, R¹⁵, R¹⁶ and R¹⁷ represent hydrogen. In a further embodiment, two of R¹³, R¹⁴, R¹⁵, R¹⁶ and R¹⁷, such as R¹⁴ and R¹⁵, independent of each other represent halo or alkyl. In a still further embodiment, R¹⁴ represents halo, such as chloro, and R¹⁵ represents alkyl, such as methyl.

In a still further special embodiment of the compound of Formula 1d, four of R¹⁸, R¹⁹, R²⁰, R²¹ and R²² represent hydrogen. In a further embodiment, one of R¹⁸, R¹⁹, R²⁰, R²¹ and R²², such as R²⁰, represents alkoxycarbonyl, such as ethoxycarbonyl.

In a further special embodiment of the compound of Formula 1d, three of R¹⁸, R¹⁹, R²⁰, R²¹ and R²² represent hydrogen. In a further embodiment, two of R¹⁸, R¹⁹, R²⁰, R²¹ and R²², such as R¹⁹ and R²⁰, independent of each other represent halo or alkoxycarbonyl, such as butoxycarbonyl. In a still further embodiment, R¹⁹ represents alkoxycarbonyl, such as butoxycarbonyl and R²⁰ represents halo such as chloro.

In a still further special embodiment of the compound of Formula 1d, -X¹-X²- represents —N═(C—R′)—, such as —N═(C—R′)— wherein R′ represents alkyl, such as methyl. In a further special embodiment, -X¹-X²- represents —NR″-(C═O)—, such as —NH—(C═O)—.

In a still further embodiment, the compound for use is a compound of Formula 1e

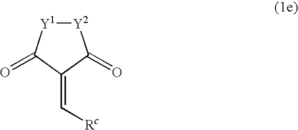

any of its stereoisomers or any mixture of its stereoisomers, or a pharmaceutically acceptable salt thereof; wherein R^{c} and —Y¹—Y²— are as defined above.

In a special embodiment of the compound of Formula 1e, R^{c} represents an optionally substituted heteroaryl group, such as optionally substituted furanyl. In a further embodiment, R^{c} represents furanyl, such as furan-2-yl.

In a further special embodiment of the compound of Formula 1e, R^{c} represents an optionally substituted aryl group, such as optionally substituted phenyl. In a further embodiment, R^{c} represents phenyl substituted with R^{e}-alkoxy and halo, such as benzyloxy and bromo. In a still further embodiment, R^{c} represents 2-benzyloxy-5-bromo-phenyl.

In a still further special embodiment of the compound of Formula 1e, —Y¹—Y²— represents

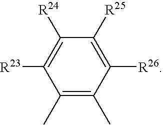

In a further embodiment, R²³, R²⁴, R²⁵ and R²⁶ represent hydrogen.

In a further special embodiment of the compound of Formula 1e, —Y¹—Y²— represents

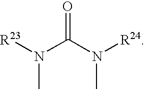

In a further embodiment, R²³ represents hydrogen. In a still further embodiment, R²⁴ represents an optionally substituted aryl group, such as halophenyl. In a further embodiment, R²⁴ represents chlorophenyl, such as 2-chlorophenyl.

In a further embodiment, the compound for use is a compound of Formula 1f

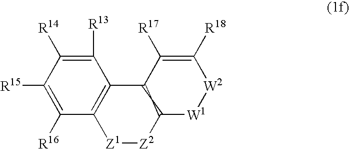

any of its stereoisomers or any mixture of its stereoisomers, or a pharmaceutically acceptable salt thereof; wherein R¹³, R¹⁴, R¹⁵, R¹⁶, R¹⁷, R¹⁸, -Z¹-Z²- and —W¹—W²— and are as defined above.

In a special embodiment of the compound of Formula 1e, R¹³, R¹⁴, R¹⁵, R¹⁶, R¹⁷, R¹⁸ independent of each other represent hydrogen or halo. In a further embodiment, all of R¹³, R¹⁴, R¹⁵, R¹⁶, R¹⁷, R¹⁸ represent hydrogen. In a still further embodiment, one of R¹³, R¹⁴, R¹⁵, R¹⁶, R¹⁷, R¹⁸, such as R¹⁴, represent halo, such as chloro.

In a further special embodiment of the compound of Formula 1e, -Z¹-Z²- represents —NR′—C(COOR″)—, such as —NH—C(COOH)—.

In a still further special embodiment of the compound of Formula 1e, -Z¹-Z²- represents or —(C═O)—(C═O)—.

In a further special embodiment of the compound of Formula 1e, —W¹—W²— represents —C(R²⁷R²⁸)—, such as —CH₂—.

In a still further special embodiment of the compound of Formula 1e, —W¹—W²— represents —CR²⁷=CR²⁸—, such as —CH═C(NO₂)—.

In a further embodiment of the compound of Formula 1e, the bond [symbol] represents a single bond. In a further embodiment, the bond [symbol] represents a double bond.

In a special embodiment, the compound of Formula 1a-1f for use is
2,3-Dibromo-5-ethoxy-6-hydroxy-benzaldehyde (a);
3-Ethoxy-2-hydroxy-benzaldehyde (b);
5-Chloro-3-ethoxy-2-hydroxy-benzaldehyde (c);
2,3-Dichloro-5-ethoxy-6-hydroxy-benzaldehyde (d);
5-Bromo-3-ethoxy-2-hydroxy-benzaldehyde (e);
6-Bromo-3-ethoxy-2-hydroxy-benzaldehyde (f);
2-Bromo-3-chloro-5-ethoxy-6-hydroxy-benzaldehyde (g);
3-Ethoxy-2-hydroxy-5-nitro-benzaldehyde (h);
((Z)-2-Cyano-3-phenyl-acryloyl)-carbamic acid ethyl ester (i);
[(Z)-2-Cyano-3-(3,4-dichlorophenyl)-acryloyl]-carbamic acid ethyl ester (j);
3-[(E)-(3-Phenyl-acryloyl)]-oxazolidin-2-one (k);
(Benzo[b]thiophene-2-carbonyl)-carbamic acid prop-2-ynyl ester (l);
4-Phenyl-3-[(E)-3-phenyl-acryloyl)]-oxazolidin-2-one (m);
(6-Bromo-2-oxo-2H-chromene-3-carbonyl)-carbamic acid ethyl ester (n);
(6,8-Dichloro-2-oxo-2H-chromene-3-carbonyl)-carbamic acid butyl ester (o);
(6,8-Diiodo-2-oxo-2H-chromene-3-carbonyl)-carbamic acid ethyl ester (p);
4-tert-Butyl-2-{[1-[5-(4-chloro-phenylazo)-2-hydroxy-phenyl]-meth-(E)-ylidene]-amino}-phenol (q);
5-(4-Bromo-phenylazo)-2-hydroxy-3-methoxy-benzaldehyde (r);
5-{5-[1-(3-Carboxy-phenyl)-3-methyl-5-oxo-1,5-dihydro-pyrazol-(4Z)-ylidene-methyl]-furan-2-yl}-2-chloro-benzoic acid butyl ester (s);
4-{5-[1-(3-Chloro-4-methyl-phenyl)-3,5-dioxo-pyrazolidin-(4Z)-ylidenemethyl]-furan-2-yl}-benzoic acid ethyl ester (t);
1-(2-Chloro-phenyl)-5-[1-furan-2-yl-meth-(E)-ylidene]-pyrimidine-2,4,6-trione (u);
2-(2-Benzyloxy-5-bromo-benzylidene)-indan-1,3-dione (v);
2-Nitro-phenanthrene-9,10-dione (w);
8-Chloro-3a,4,5,9b-tetrahydro-3H-cyclopenta[c]quinoline-4-carboxylic acid (x);
   or a pharmaceutically acceptable salt thereof.

In a further embodiment, the compound of Formula 1a or 1b for use is a compound of the invention as described below.

### Compounds of the Invention

A further aspect of the invention provides compounds of Formula 1a or 1b:

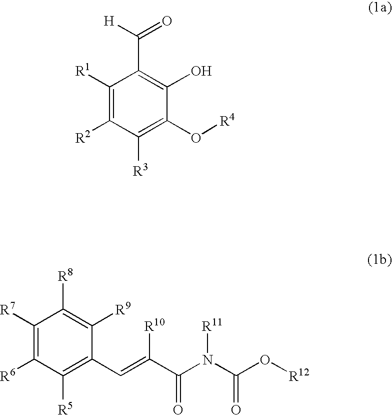

any of its stereoisomers or any mixture of its stereoisomers,
or a pharmaceutically acceptable salt thereof;
wherein
one of R¹, R² and R³ represents —(C≡C)ₙ—R^{a}; wherein
wherein n is 0 or 1; and
R^{a} represents an aryl or a heteroaryl group;
   which aryl or heteroaryl group is optionally substituted with one or more substituents independently selected from the group consisting of:
      halo, trifluoromethyl, trifluoromethoxy, cyano, nitro, hydroxy, alkoxy, cycloalkoxy, alkoxyalkyl, cycloalkoxyalkyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
         wherein R′ and R″ independent of each other are hydrogen or alkyl;
            the remaining two of R¹, R² and R³ are independently selected from the group consisting of:
hydrogen, halo, trifluoromethyl, trifluoromethoxy, cyano, nitro, alkyl, hydroxy, alkoxy, formyl and alkylcarbonyl;
   R⁴ represents hydrogen or alkyl;
   R⁵, R⁶, R⁷ and R⁸ are independently selected from the group consisting of:
hydrogen, halo, trifluoromethyl, trifluoromethoxy, cyano, nitro, alkyl, hydroxy, alkoxy or an aryl or a heteroaryl group;
   which aryl or heteroaryl group is optionally substituted with one or more substituents independently selected from the group consisting of:
      halo, trifluoromethyl, trifluoromethoxy, cyano, nitro, hydroxy, alkoxy, cycloalkoxy, alkoxyalkyl, cycloalkoxyalkyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
         wherein R′ and R″ independent of each other are hydrogen or alkyl;
            R⁹ and R¹⁰ together form —(O—(C═O))—, —O— or —S—; or
            R⁹ represents hydrogen or alkyl; and R¹⁰ represents hydrogen, cyano or alkyl;
            R¹¹ and R¹² together form —(CHR′—CH₂)—;
wherein R′ represents hydrogen, alkyl or phenyl; or
   R¹¹ represents hydrogen or alkyl; and
   R¹² represents hydrogen, alkyl, alkenyl or alkynyl;
which alkyl, alkenyl or alkynyl is optionally substituted with an aryl or heteroaryl group;
   which aryl or heteroaryl group is optionally substituted with one or more substituents independently selected from the group consisting of:
      halo, trifluoromethyl, trifluoromethoxy, cyano, nitro, hydroxy, alkoxy, cycloalkoxy, alkoxyalkyl, cycloalkoxyalkyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
         wherein R′ and R″ independent of each other are hydrogen or alkyl.

In a special embodiment, the compound of Formula 1a or 1b is a compound of Formula 1a

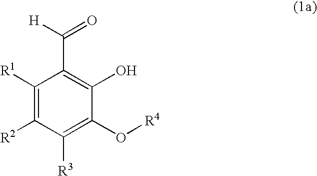

any of its stereoisomers or any mixture of its stereoisomers, or a pharmaceutically acceptable salt thereof; wherein R¹, R², R³ and R⁴ are as defined above. In a further embodiment, one of R¹, R² and R³ represents —C≡C—R^{a}. In a still further embodiment, one of R¹, R² and R³ represents a monocyclic heteroaryl group.

In a further embodiment, the compound of Formula 1a or 1b is a compound of Formula 1b

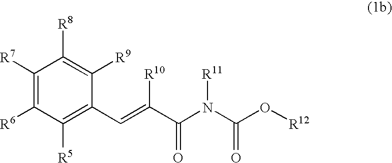

any of its stereoisomers or any mixture of its stereoisomers, or a pharmaceutically acceptable salt thereof; wherein R⁵, R⁶, R⁷, R⁸, R⁹, R¹⁰, R¹¹ and R¹² are as defined above. In a further embodiment, R¹² represents substituted alkynyl. In a still further embodiment, R⁹ represents hydrogen and R¹⁰ represents cyano.

In a still further embodiment, the compound of formula 1b is a compound of Formula 1b1, 1b2, 1b3 or 1b4:

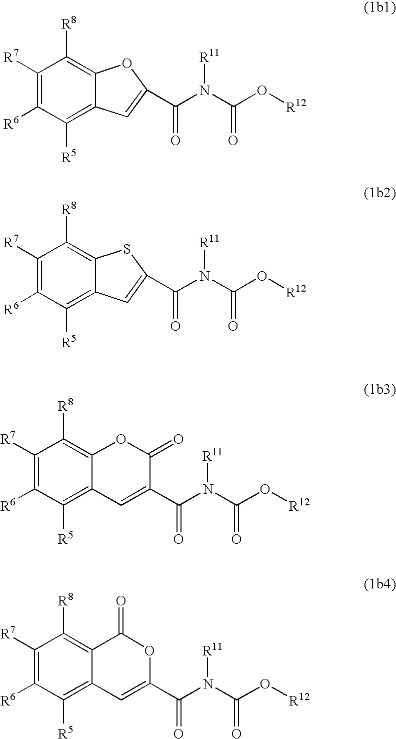

any of its stereoisomers or any mixture of its stereoisomers,
or a pharmaceutically acceptable salt thereof;
wherein one of R⁵, R⁶, R⁷ and R⁸ is an optionally substituted aryl or a heteroaryl group; and the remaining three of R⁵, R⁶, R⁷ and R⁸ and R¹¹ and R¹² are as defined above.

In a special embodiment, the compound of Formula 1a or 1b is
4-Ethoxy-3-hydroxy-biphenyl-2-carbaldehyde;
4-Ethoxy-2′-fluoro-3-hydroxy-biphenyl-2-carbaldehyde;
4-Ethoxy-3′-fluoro-3-hydroxy-biphenyl-2-carbaldehyde;
4-Ethoxy-4′-fluoro-3-hydroxy-biphenyl-2-carbaldehyde;
4-Ethoxy-3-hydroxy-2′-methoxy-biphenyl-2-carbaldehyde;
4-Ethoxy-3-hydroxy-3′-methoxy-biphenyl-2-carbaldehyde;
4-Ethoxy-3-hydroxy-4′-methoxy-biphenyl-2-carbaldehyde;
3-Ethoxy-2-hydroxy-6-pyridin-3-yl-benzaldehyde;
3-Ethoxy-6-furan-2-yl-2-hydroxy-benzaldehyde;
3-Ethoxy-6-furan-3-yl-2-hydroxy-benzaldehyde;
3-Ethoxy-2-hydroxy-6-thiophen-2-yl-benzaldehyde;
3-Ethoxy-2-hydroxy-6-thiophen-3-yl-benzaldehyde;
5-Ethoxy-4-hydroxy-biphenyl-3-carbaldehyde;
3-Ethoxy-5-furan-2-yl-2-hydroxy-benzaldehyde;
3-Ethoxy-5-furan-3-yl-2-hydroxy-benzaldehyde;
3-Ethoxy-2-hydroxy-5-thiophen-2-yl-benzaldehyde;
3-Ethoxy-2-hydroxy-5-thiophen-3-yl-benzaldehyde;
6-Chloro-4-ethoxy-3-hydroxy-biphenyl-2-carbaldehyde;
((Z)-3-Biphenyl-3-yl-2-cyano-acryloyl)-carbamic acid isopropyl ester;
[(Z)-2-Cyano-3-(3-furan-2-yl-phenyl)-acryloyl]-carbamic acid isopropyl ester;
((Z)-3-Biphenyl-4-yl-2-cyano-acryloyl)-carbamic acid ethyl ester;
[(Z)-2-Cyano-3-(4-furan-2-yl-phenyl)-acryloyl]-carbamic acid ethyl ester;
(2-Oxo-6-phenyl-2H-chromene-3-carbonyl)-carbamic acid ethyl ester;
(6-Furan-2-yl-2-oxo-2H-chromene-3-carbonyl)-carbamic acid ethyl ester;
(6-Furan-3-yl-2-oxo-2H-chromene-3-carbonyl)-carbamic acid ethyl ester;
(2-Oxo-6-thiophen-2-yl-2H-chromene-3-carbonyl)-carbamic acid ethyl ester;
(2-Oxo-6-thiophen-3-yl-2H-chromene-3-carbonyl)-carbamic acid ethyl ester;
3-Ethoxy-2-hydroxy-6-phenylethynyl-benzaldehyde;
3-Ethoxy-2-hydroxy-6-pyridin-3-ylethynyl-benzaldehyde;
3-Ethoxy-2-hydroxy-6-(4-methoxy-phenylethynyl)-benzaldehyde;
3-Ethoxy-2-hydroxy-6-thiophen-3-ylethynyl-benzaldehyde;
3-Ethoxy-2-hydroxy-5-phenylethynyl-benzaldehyde;
3-Chloro-5-ethoxy-6-hydroxy-2-phenylethynyl-benzaldehyde;
(Benzo[b]thiophene-2-carbonyl)-carbamic acid phenylethynyl ester;
(Benzo[b]thiophene-2-carbonyl)-carbamic acid furan-2-ylethynyl ester;
(Benzo[b]thiophene-2-carbonyl)-carbamic acid thiophen-2-ylethynyl ester;
(Benzo[b]thiophene-2-carbonyl)-carbamic acid furan-3-ylethynyl ester;
(Benzo[b]thiophene-2-carbonyl)-carbamic acid thiophen-3-ylethynyl ester;
[(Z)-2-Cyano-3-(3,4-dichloro-phenyl)-acryloyl]-carbamic acid methyl ester;
[(Z)-2-Cyano-3-(3,4-dichloro-phenyl)-acryloyl]-carbamic acid isopropyl ester;
[(Z)-2-Cyano-3-(3,4-dichloro-phenyl)-acryloyl]-carbamic acid propyl ester;
((Z)-2-Cyano-3-phenyl-acryloyl)-carbamic acid methyl ester;
((Z)-2-Cyano-3-phenyl-acryloyl)-carbamic acid isopropyl ester;
(Benzo[b]thiophene-2-carbonyl)-carbamic acid ethyl ester;
(Benzo[b]thiophene-2-carbonyl)-carbamic acid vinyl ester;
   or a pharmaceutically acceptable salt thereof.

Any combination of two or more of the embodiments as described above is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contains of from one to six carbon atoms (C₁₋₆-alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkenyl group designates a carbon chain containing one or more double bonds, including di-enes, tri-enes and poly-enes. In a preferred embodiment the alkenyl group of the invention comprises of from two to six carbon atoms (C₂₋₆-alkenyl), including at least one double bond. In a most preferred embodiment the alkenyl group of the invention is ethenyl; 1- or 2-propenyl; 1-, 2- or 3-butenyl, or 1,3-butadienyl; 1-, 2-, 3-, 4- or 5-hexenyl, or 1,3-hexadienyl, or 1,3,5-hexatrienyl.

In the context of this invention an alkynyl group designates a carbon chain containing one or more triple bonds, including di-ynes, tri-ynes and poly-ynes. In a preferred embodiment the alkynyl group of the invention comprises of from two to six carbon atoms (C₂₋₆-alkynyl), including at least one triple bond. In its most preferred embodiment the alkynyl group of the invention is ethynyl; 1-, or 2-propynyl; 1-, 2-, or 3-butynyl, or 1,3-butadiynyl; 1-, 2-, 3-, 4-pentynyl, or 1,3-pentadiynyl; 1-, 2-, 3-, 4-, or 5-hexynyl, or 1,3-hexadiynyl or 1,3,5-hexatriynyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

Alkoxy is O-alkyl, wherein alkyl is as defined above.

Cycloalkoxy means O-cycloalkyl, wherein cycloalkyl is as defined above.

Cycloalkylalkyl means cycloalkyl as above and alkyl as above, meaning for example, cyclopropylmethyl.

In the context of this invention an aryl group designates a carbocyclic aromatic ring system such as phenyl, naphthyl (1-naphthyl or 2-naphthyl) or fluorenyl.

In the context of this invention a heteroaryl group designates an aromatic mono- or bicyclic heterocyclic group, which holds one or more heteroatoms in its ring structure. Preferred heteroatoms include nitrogen (N), oxygen (O), and sulphur (S).

Preferred monocyclic heteroaryl groups of the invention include aromatic 5- and 6-membered heterocyclic monocyclic groups, including for example, but not limited to, oxazolyl, isoxazolyl, thiazolyl, isothiazolyl, tetrazolyl, 1,2,4-oxadiazolyl, 1,2,5-oxadiazolyl, 1,3,4-oxadiazolyl, triazolyl, 1,2,4-thiadiazolyl, 1,2,5-thiadiazolyl, imidazolyl, pyrrolyl, pyrazolyl, furanyl, thienyl, pyridyl, pyrimidyl, or pyridazinyl.

Preferred bicyclic heteroaryl groups of the invention include for example, but not limited to, indolizinyl, indolyl, isoindolyl, indazolyl, benzofuranyl, benzo[b]thienyl, benzimidazolyl, benzoxazolyl, benzooxadiazolyl, benzothiazolyl, benzo[d]isothiazolyl, purinyl, quinolinyl, isoquinolinyl, cinnolinyl, phthalazinyl, quinazolinyl, quinoxalinyl, 1,8-naphthyridinyl, pteridinyl, and indenyl.

### Pharmaceutically Acceptable Salts

The chemical compound or the compound for use according to the invention may be provided in any form suitable for the intended administration. Suitable forms include pharmaceutically (i.e. physiologically) acceptable salts, and pre- or prodrug forms of the chemical compound or the compound for use according to the invention.

Examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound or the compound for use according to the invention include, without limitation, the sodium, the potassium, the calcium, the magnesium, the zinc, the aluminium, the lithium, the choline, the lysinium, and the ammonium salt, and the like, of a chemical compound or the compound for use according to the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound or the compound for use according to the invention include examples of suitable prodrugs of the substances for use or according to the invention including compounds modified at one or more reactive or derivatizable groups of the parent compound. Of particular interest are compounds modified at a carboxyl group, a hydroxyl group, or an amino group. Examples of suitable derivatives are esters or amides.

The chemical compound or the compound for use according to the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds or the compounds for use according to the present invention may exist in different stereoisomeric forms—including enantiomers, diastereomers and cis-trans-isomers.

The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid—by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphor-sulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials or intermediates.

### Labelled Compounds

The compounds or the compounds for use according to the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds or the compounds for use according to the invention may be useful as diagnostic tools, radio tracers, or monitoring agents in various diagnostic methods, and for in vivo receptor imaging.

The labelled compound or the compound for use according to the invention preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹³¹I, ¹²⁵I, ¹²³I, and ¹⁸F.

The physical method for detecting the labelled compound of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

Some of the compounds for use according to the invention are known compounds that are commercially available.

Other chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

Compounds of the invention or for use according to the invention may be tested for their ability to modulate a PDZ domain, such as the PDZ domain of PICK1 e.g. as described in the “TEST METHODS” paragraph. Further, in particular in relation to pain disorders, compounds of the invention or for use according to the invention may be tested in various in vivo pain models well known in the art, such as the hot plate test, the formalin test, capsaicin-induced sensitization, the CFA test, the CCI test and the SNI model.

Based on the PICK1 inhibition and PICK1 interaction with GluR2 the compounds of the invention or for use according to the invention are considered useful for the treatment, prevention or alleviation of a disease or a disorder or a condition of a mammal, including a human, which disease, disorder or condition is responsive to modulation of a PDZ domain. In a special embodiment, the disease or disorder or condition is responsive to modulation of a PDZ domain is disease or disorder or condition is responsive to modulation of the PDZ domain of PICK1.

In a further embodiment, the compounds of the invention are considered useful for the treatment, prevention or alleviation of a variety of disorders of the CNS and PNS and disorders of other origin, including acute pain, chronic pain, neuropathic pain, intractable pain, migraine, neurological and psychiatric disorders, depression, anxiety, psychosis, schizophrenia, excitatory amino acid-dependent psychosis, cognitive disorders, dementia, senile dementia, AIDS-induced dementia, stress-related psychiatric disorders, stroke, global ischaemic, focal ischaemic, haemorrhagic stroke, cerebral hypoxia, cerebral ischaemia, cerebral infarction, cerebral ischaemia resulting from thromboembolic or haemorrhagic stroke, cardiac infarction, brain trauma, brain oedema, cranial trauma, brain trauma, spinal cord trauma, bone-marrow lesions, hypoglycaemia, anoxia, neuronal damage following hypoglycaemia, hypotonia, hypoxia, perinatal hypoxia, cardiac arrest, acute neurodegenerative diseases or disorders, chronic neurodegenerative diseases or disorders, brain ischaemia, CNS degenerative disorders, Parkinson's disease, Alzheimer's disease, Huntington's disease, idiopathic Parkinson's Disease, drug induced Parkinson's Disease, amyotrophic lateral sclerosis (ALS), post-acute phase cerebral lesions, chronic diseases of the nervous system, cerebral deficits subsequent to cardiac bypass surgery, cerebral deficits subsequent to grafting, perinatal asphyxia, anoxia from drowning, anoxia from pulmonary surgery. anoxia from cerebral trauma, hypoxia induced nerve cell damage, epilepsy, status epilepticus, seizure disorders, cerebral vasospasm, CNS mediated spasms, motility disorders, muscular spasms, urinary incontinence, convulsions, disorders responsive to anticonvulsants, autoimmune diseases, emesis, nausea, obesity, chemical dependencies, chemical addictions, addictions, withdrawal symptoms, drug induced deficits, alcohol induced deficits, drug addiction, ocular damage, retinopathy, retinal neuropathy, tinnitus, and tardive dyskinesia.

In a special embodiment, the compounds of the invention are considered useful for the treatment, prevention or alleviation of: pain, acute pain, chronic pain, neuropathic pain, intractable pain, inflammatory pain, neurogenic pain, fibromyalgia, chronic fatigue syndrome, nociceptive pain, cancer pain, postoperative pain, migraine, tension-type headache, pain during labour and delivery, breakthrough pain, stroke, drug abuse and cocaine abuse.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to modulation of a PDZ domain, and which method comprises administering to such a living animal body, including a human, in need thereof an effective amount of a chemical compound of the invention or for use according to the invention.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

### BRIEF DESCRIPTION OF THE DRAWINGS

The present invention is further illustrated by reference to the accompanying drawing, in which:

FIG. 1 shows PICK1 saturation binding. Fluorescently labeled peptides (40 nm) corresponding to the C-terminal 13 residues of DAT, PKC_{α}13, and β₂AR (DAT13 OrG, PKC_{α}13 OrG, and β₂AR13 OrG) were titrated with increasing amounts of purified WT PICK1 protein. After 15 min of incubation, FP values were determined as a direct read-out of peptide binding to PICK1. Data are representative of at least five similar experiments.

FIG. 2 shows PICK1 competition binding. A, competition binding to PICK1 of Oregon Green-labeled DAT peptide (DAT13 OrG) with unlabeled peptides corresponding to the 13 C-terminal residues of DAT, PKC_{α}, and β₂AR. B, competition binding to PICK1 of Oregon Green-labeled DAT peptide (DAT13 OrG) with unlabeled peptides corresponding to the 13 C-terminal residues of GluR2. Data are representative of at least three similar experiments. For the experiments, tracer (40 nM) was incubated with a fixed subsaturating amount of PICK1 and titrated with increasing amounts of the indicated unlabeled peptides. After 15 min of incubation, FP values were determined.

FIG. 3 shows screening setup and identification of an active well. 1B-C: Oregon Green-labeled DAT peptide (40 nM) was incubated with a fixed subsaturating amount of PICK1 to estimate top of the window. 1 D-E: Oregon Green-labeled DAT peptide (40 nM) alone to estimate bottom of window. 1F-G: Oregon Green-labeled DAT peptide (40 nM) was incubated with a fixed subsaturating amount of PICK1 and 20 μM of non-labeled DAT as a positive control for competition. 2A-12H: Oregon Green-labeled DAT peptide (40 nM) was incubated with a fixed subsaturating amount of PICK1 and 100 μM of the small molecule drug to be screened. All wells contained 10% DMSO. After 15 min of incubation, FP values were determined. ‘Active wells’ (FP<80% of 1B-C), wells showing increased FP and wells showing increased fluorescence (indicative of fluorescent drugs) are highlighted.

FIG. 4 shows PICK1 competition binding. Competition binding to PICK1 of Oregon Green-labeled DAT peptide (DAT13 OrG) with compound (j). Data are representative of at least three similar experiments. For the experiments, tracer (40 nM) was incubated with a fixed subsaturating amount of PICK1 and titrated with increasing amounts of compound (j). After 15 min of incubation, FP values were determined. The Kⱼ of compound (j) was shown to be 40 μM [39.3-41.7 μM].

FIG. 5A-E show additional competition curves for five additional compounds. Competition binding to PICK1 of Oregon Green-labeled DAT peptide (DAT13 OrG). Data are represent-tative of at least three similar experiments. For the experiments, tracer (40 nM) was incubated with a fixed subsaturating amount of PICK1 and titrated with increasing amounts of compound. After 15 min of incubation, FP values were determined.

FIG. 6 shows representative experiment showing the inhibition of GST-DAT pull-down of PICK by the compound compound (j) and the peptide GIT1b.

FIG. 7 shows densitometry analysis of GST-DAT pull-down assay of PICK1. Indicated compounds or the C-terminal peptide of GIT1b were added at a concentration of 50 μM prior to performing the pull-down. Data are means±SE of three independent experiments and expressed in percent of the pull-down seen in the presence of vehicle.

FIG. 8 shows that FRET can be observed between eCFP-GluR2 C29 and eYFP-PICK1. Indicated constructs were expressed transiently in COS7 cells and FRET values measured as described in Methods. Data are means±S.E. of N=15−46.

FIGS. 9A and 9B shows FRET between eCFP-GluR2 C29 and eYFP-PICK1 was specifically reduced by compound (j). Indicated constructs were expressed transiently in COS7 cells and FRET values measured as described in Methods. Compound or vehicle (1% DMASO) was added 20 min prior to image acquisition. Data are means±S.E. of N=8−41, **p<0.01 as compared to vehicle.

### EXAMPLES

The invention is further illustrated with reference to the following examples, which are not intended to be in any way limiting to the scope of the invention as claimed.

### Synthetic Examples

All reactions involving air sensitive reagents or intermediates are performed under nitrogen and in anhydrous solvents. Sodium sulphate is used as drying agent in the workup-procedures and solvents are evaporated under reduced pressure.

### Method A

### 4-Ethoxy-3-hydroxy-biphenyl-2-carbaldehyde

Is prepared by the Suzuki-reaction, by stirring a mixture of 6-bromo-3-ethoxy-2-hydroxy-benzaldehyde, benzenebononic acid (1.5 eq), Pd(PPh₃)₄ (2%), 1,3-dihydroxypropane (3 eq), potassium carbonate (3 eq), water and DMT at reflux for 15 h. Water is added and the mixture is extracted twice with dichloromethane.

Chromatography on silica gel with dichloromethane and methanol solvent gives the title compound.

### 4-Ethoxy-2′-fluoro-3-hydroxy-biphenyl-2-carbaldehyde

Is prepared according to method A from 2-fluorobenzeneboronic acid.

### 4-Ethoxy-3′-fluoro-3-hydroxy-biphenyl-2-carbaldehyde

Is prepared according to method A from 3-fluorobenzeneboronic acid.

### 4-Ethoxy-4′-fluoro-3-hydroxy-biphenyl-2-carbaldehyde

Is prepared according to method A from 4-fluorobenzeneboronic acid.

### 4-Ethoxy-3-hydroxy-2′-methoxy-biphenyl-2-carbaldehyde

Is prepared according to method A from 2-methoxybenzeneboronic acid.

### 4-Ethoxy-3-hydroxy-3′-methoxy-biphenyl-2-carbaldehyde

Is prepared according to method A from 3-methoxybenzeneboronic acid.

### 4-Ethoxy-3-hydroxy-4′-methoxy-biphenyl-2-carbaldehyde

Is prepared according to method A from 4-methoxybenzeneboronic acid.

### 3-Ethoxy-2-hydroxy-6-pyridin-3-yl-benzaldehyde

Is prepared according to method A from diethyl-3-pyridylborane.

### 3-Ethoxy-6-furan-2-yl-2-hydroxy-benzaldehyde

Is prepared according to method A from 2-furaneboronic acid.

### 3-Ethoxy-6-furan-3-yl-2-hydroxy-benzaldehyde

Is prepared according to method A from 3-furaneboronic acid.

### 3-Ethoxy-2-hydroxy-6-thiophen-2-yl-benzaldehyde

Is prepared according to method A from 2-thiopheneeboronic acid.

### 3-Ethoxy-2-hydroxy-6-thiophen-3-yl-benzaldehyde

Is prepared according to method A from 3-thiopheneboronic acid.

### 5-Ethoxy-4-hydroxy-biphenyl-3-carbaldehyde

Is prepared from 5-bromo-3-ethoxy-2-hydroxy-benzaldehyde and benzeneboronic acid according to method A.

### 3-Ethoxy-5-furan-2-yl-2-hydroxy-benzaldehyde

Is prepared from 5-bromo-3-ethoxy-2-hydroxy-benzaldehyde and 2-furanboronic acid according to method A.

### 3-Ethoxy-5-furan-3-yl-2-hydroxy-benzaldehyde

Is prepared from 5-bromo-3-ethoxy-2-hydroxy-benzaldehyde and 3-furanboronic acid according to method A.

### 3-Ethoxy-2-hydroxy-5-thiophen-2-yl-benzaldehyde

Is prepared from 5-bromo-3-ethoxy-2-hydroxy-benzaldehyde and 2-thiophenboronic acid according to method A.

### 3-Ethoxy-2-hydroxy-5-thiophen-3-yl-benzaldehyde

Is prepared from 5-bromo-3-ethoxy-2-hydroxy-benzaldehyde and 3-thiophenboronic acid according to method A.

### 6-Chloro-4-ethoxy-3-hydroxy-biphenyl-2-carbaldehyde

Is prepared from 2-bromo-3-chloro-5-ethoxy-6-hydroxy-benzaldehyde and benzeneboronic acid according to method A.

### ((Z)-3-Biphenyl-3-yl-2-cyano-acryloyl)-carbamic acid isopropyl ester

Is prepared according to method A from [(Z)-3-(3-bromo-phenyl)-2-cyano-acryloyl]-carbamic acid ethyl ester and benzeneboronic acid.

### [(Z)-2-Cyano-3-(3-furan-2-yl-phenyl)-acryloyl]-carbamic acid isopropyl ester

Is prepared according to method A from [(Z)-3-(3-bromo-phenyl)-2-cyano-acryloyl]-carbamic acid ethyl ester and 2-furanboronic acid.

### ((Z)-3-Biphenyl-4-yl-2-cyano-acryloyl)-carbamic acid ethyl ester

Is prepared according to method A from [(Z)-3-(4-bromo-phenyl)-2-cyano-acryloyl]-carbamic acid ethyl ester and benzeneboronic acid.

### [(Z)-2-Cyano-3-(4-furan-2-yl-phenyl)-acryloyl]-carbamic acid ethyl ester

Is prepared according to method A from [(Z)-3-(4-bromo-phenyl)-2-cyano-acryloyl]-carbamic acid ethyl ester and 2-furanboronic acid.

### (2-Oxo-6-phenyl-2H-chromene-3-carbonyl)-carbamic acid ethyl ester

Is prepared according to method A from (6-bromo-2-oxo-2H-chromene-3-carbonyl)-carbamic acid ethyl ester and benzeneboronic acid.

### (6-Furan-2-yl-2-oxo-2H-chromene-3-carbonyl)-carbamic acid ethyl ester

Is prepared according to method A from (6-bromo-2-oxo-2H-chromene-3-carbonyl)-carbamic acid ethyl ester and 2-furanboronic acid.

### (6-Furan-3-yl-2-oxo-2H-chromene-3-carbonyl)-carbamic acid ethyl ester

Is prepared according to method A from (6-bromo-2-oxo-2H-chromene-3-carbonyl)-carbamic acid ethyl ester and 3-furanboronic acid.

### (2-Oxo-6-thiophen-2-yl-2H-chromene-3-carbonyl)-carbamic acid ethyl ester

Is prepared according to method A from (6-bromo-2-oxo-2H-chromene-3-carbonyl)-carbamic acid ethyl ester and 2-furanboronic acid.

### (2-Oxo-6-thiophen-3-yl-2H-chromene-3-carbonyl)-carbamic acid ethyl ester

Is prepared according to method A from (6-bromo-2-oxo-2H-chromene-3-carbonyl)-carbamic acid ethyl ester and 3-furanboronic acid.

### Method B

### 3-Ethoxy-2-hydroxy-6-phenylethynyl-benzaldehyde

Is prepared by the Sonogashira reaction, by stirring a mixture of 6-bromo-3-ethoxy-2-hydroxy-benzaldehyde, phenylacetylene (3 eq), diisopropylethylamine (2 eq), CuI (0.1 eq), Pd (PPh₃)₄ (2%) and dioxane at reflux for 15 h. Water is added and the mixture is extracted twice with dichloromethane. Chromatography on silica gel with dichloromethane and methanol solvent gives the title compound.

### 3-Ethoxy-2-hydroxy-6-pyridin-3-ylethynyl-benzaldehyde

Is prepared according to method B from 3-pyridylacetylene.

### 3-Ethoxy-2-hydroxy-6-(4-methoxy-phenylethynyl)-benzaldehyde

Is prepared according to method B from 4-methoxyphenylacetylene.

### 3-Ethoxy-2-hydroxy-6-thiophen-3-ylethynyl-benzaldehyde

Is prepared according to method B from 3-thienylacetylene.

### 3-Ethoxy-2-hydroxy-5-phenylethynyl-benzaldehyde

Is prepared according to method B from 5-Bromo-3-ethoxy-2-hydroxy-benzaldehyde.

### 3-Chloro-5-ethoxy-6-hydroxy-2-phenylethynyl-benzaldehyde

Is prepared according to method B from 2-bromo-3-chloro-5-ethoxy-6-hydroxy-benzaldehyde.

### (Benzo[b]thiophene-2-carbonyl)-carbamic acid phenylethynyl ester

Is prepared according to method B from (Benzo[b]thiophene-2-carbonyl)-carbamic acid ethynyl and iodobenzene.

### (Benzo[b]thiophene-2-carbonyl)-carbamic acid furan-2-ylethynyl ester

Is prepared according to method B from (Benzo[b]thiophene-2-carbonyl)-carbamic acid ethynyl and 2-bromofuran.

### (Benzo[b]thiophene-2-carbonyl)-carbamic acid thiophen-2-ylethynyl ester

Is prepared according to method B from (Benzo[b]thiophene-2-carbonyl)-carbamic acid ethynyl and 2-bromothiophene

### (Benzo[b]thiophene-2-carbonyl)-carbamic acid furan-3-ylethynyl ester

Is prepared according to method B from (Benzo[b]thiophene-2-carbonyl)-carbamic acid ethynyl and 3-bromofuran

### (Benzo[b]thiophene-2-carbonyl)-carbamic acid thiophen-3-ylethynyl ester

Is prepared according to method B from (Benzo[b]thiophene-2-carbonyl)-carbamic acid ethynyl and 3-bromothiophene

### Method C

### [(Z)-2-Cyano-3-(3,4-dichloro-phenyl)-acryloyl]-carbamic acid methyl ester

Is prepared by solvolysis under acidic conditions from [(Z)-2-Cyano-3-(3,4-dichloro-phenyl)-acryloyl]-carbamic acid ethyl ester and methanol.

### [(Z)-2-Cyano-3-(3,4-dichloro-phenyl)-acryloyl]-carbamic acid isopropyl ester

Is prepared according to method C from [(Z)-2-Cyano-3-(3,4-dichloro-phenyl)-acryloyl]-carbamic acid ethyl ester and 2-propanol.

### [(Z)-2-Cyano-3-(3,4-dichloro-phenyl)-acryloyl]-carbamic acid propyl ester

Is prepared according to method C from [(Z)-2-Cyano-3-(3,4-dichloro-phenyl)-acryloyl]-carbamic acid ethyl ester and n-propanol.

### ((Z)-2-Cyano-3-phenyl-acryloyl)-carbamic acid methyl ester

Is prepared according to method C from ((Z)-2-Cyano-3-phenyl-acryloyl)-carbamic acid ethyl ester and methanol.

### ((Z)-2-Cyano-3-phenyl-acryloyl)-carbamic acid isopropyl ester

Is prepared according to method C from ((Z)-2-Cyano-3-phenyl-acryloyl)-carbamic acid ethyl ester and 2-propanol.

### Method D

### (Benzo[b]thiophene-2-carbonyl)-carbamic acid ethyl ester

Is prepared from (benzo[b]thiophene-2-carbonyl)-carbamic acid ethynyl ester by stirring with Pd/C under an atmosphere of hydrogen. Workup: filtration through celite.

### (Benzo[b]thiophene-2-carbonyl)-carbamic acid vinyl ester

Is prepared from benzo[b]thiophene-2-carbonyl)-carbamic acid ethynyl, using Lindlars poisoned Pd-catalyst.

### Test Methods

### Purification of PICK1 for Fluorescence Polarization Assay

The entire coding region of rat PICK1 (residues 2-416) is amplified from a pCINEO vector by PCR using pfu polymerase according to the instructions by the manufacturer (Stratagene, La Jolla, Calif.). The primers used introduce a 5′ restriction site for MunI and 3′ restriction site for AvrII. The PCR fragment is cleaved with MunI and AvrII and cloned into the reading frame of the pET41a vector (Novagen, Madison, Wis.) producing an N-terminally glutathione-S-transferase (GST) fusion of PICK1. The GST-PICK1 fusion was expressed in E. coli BL21(DE3) pLysS (Novagen). The transformed bacteria are grown to OD₆₀₀ 0.6 and expression of the fusion protein is induced with isopropyl-β-D1-thiogalactopyranoside (105 μM) overnight at 30° C. The bacteria are lysed by freezing and thawing in TBS buffer containing [50 mM Tris pH 7.4, 125 mM NaCl, 1% TX-100, 20 μg/mL DNAse I, 1 mM DTT (Sigma)]. The lysate is cleared by centrifugation (rotor SS-34, 18000 rpm, 48000×g, 30 min). The supernatant is incubated with glutathione-sepharose beads (Amersham Biosciences) under slow rotation for 90 minutes at 4° C. The beads are pelleted at 3500 g for 5 minutes and washed in TBS buffer containing [50 mM Tris pH 7.4, 125 mM NaCl, 0.1% TX-100, 1 mM DTT] by three batch washes. The protein is separated from the GST domain by cleavage with thrombin protease (Novagen) in the above wash buffer at 4° C. overnight. Samples of 10 μl are taken from the protein solution for determination of protein concentration and for analysis by SDS-PAGE. Protein determinations are performed using the BCA Protein Assay Reagent kit (Pierce Biotechnology, Inc, Rockford, Ill.) according to manufactures protocol using bovine serum albumin as standard. Gels are stained with GelCode Blue Stain Reagent (Pierce Biotechnology) in order to inspect size, integrity and purity of the protein (Madsen et al., JBC, 280, 20539-48, 2005).

### Fluorescence Polarization Assay

This assay is used to screen for compounds binding to the PDZ binding groove in PICK1. The assay is based on the predicted decrease in rotational diffusion of a fluorescently labeled peptide upon its binding to a larger protein. The decrease in rotational diffusion upon binding of a fluorescent labeled peptide to PICK1 can be detected as an increase in fluorescence polarization (FP). Binding of a small-molecule compound to the PDZ binding groove can be detected by its ability to displace the fluorescently labeled peptide resulting in a decreased in FP (Madsen et al., JBC, 280, 20539-48, 2005).

To perform the assay, compounds are loaded (10 μl of each) in microtiter-plates (88 compounds per 96 well plate) (Corning) at a concentration of 1 mM in DMSO. To each well is added 90 μl buffer containing Tris base (50 mM), NaCl (125 mM), TX-100 (0.1%), PICK1 (0.45 μM) and fluorescently labeled peptide. This peptide corresponds to the C-terminus of a protein that binds to the PICK1 PDZ domain. For example, the peptide can correspond to the last 13 C-terminal residues of the DAT, of protein kinase Cα or of the GluR2 subunit of the AMPA reaceptor. The peptide can be labeled with any fluorophore. For example it can be labeled with a sulfhydryl-reactive derivative of Oregon Green via a cysteine introduced in the peptide. The peptide is used in a concentration of about 40 nM. The final concentration of the compounds in the initial screen is 100 μM. After approximately 15 min of incubation at 3° C. the plates are analyzed in a Chameleon plate-reader (HIDEX) in the FP mode using a 488 nm excitation filter and a 535 nm long pass emission filter. Each measurement is an average of 100 flashes and is carried out four times. FP is calculated according to the equation FP=(I_{V}−g*I_{H})/(I_{V}+g*I_{H}). As negative and positive controls, respectively, pure DMSO and DMSO with unlabeled DAT peptide (20 μM final conc.) are measured in parallel. All compounds are tested twice on separate plates. Active compounds are recognized by a decrease in (below 80%) depolarization compared to the control wells with pure DMSO.

To determine the affinity of identified compounds (Kᵢ values) the K_{d} value for the fluorescently labeled peptide is first determined by performing saturation binding isotherms using a fixed amount of Oregon Green labeled peptide (40 nM) with an increasing amount of PICK1 in a final volume of 100 μl. An equilibrium saturation binding isotherms is constructed by plotting FP versus the concentration of PICK1. To determine K_{d}, a curve is fitted with the equation Y=FP_{b}*X/(K_{d}*X), where FP_{b} is the maximal value of FP reached by complete saturation. Subsequently, competition binding experiments are carried out in the same format as the saturation binding experiments using a fixed concentration of fluorescently labeled peptide (40 nM) and a fixed non-saturating concentration of purified PICK1, and an increasing concentration of the compounds to be tested (up to 1 mM). Equilibrium competition binding isotherms are constructed by plotting FP versus the concentration of compound. To determine Kᵢ, a curve was fitted to the equation FP=FP_{f}+((FP_{f}−FP_{b})*[Rₜ])/(K_{d}*(1+X/Kᵢ)+[Rₜ]), with FP_{f} and FP_{b} being the FP value of the free and bound peptide, [Rₜ] the concentration of PICK1 and K_{d} the apparent dissociation constant determined from parallel saturation experiments. Kᵢ, FP_{b}, and FP_{f} were treated as free parameters.

### Pull Down

To verify binding of active compounds identified in the FP screen, a biochemical pulldown assay can be employed.

A fusion between GST and the 24 C-terminal amino acids of the dopamine transporter (GST DAT C24) is expressed in BL21(DE3)pLysS (Novagen) using the pET41a vector (Novagen, Madison, Wis.) and purified as described for PICK1 but without digestion with thrombin.

5 μl dry volume of glutathione-coated sepharose beads (Pharmacia) with bound GST alone or GST DAT C24 are suspended in 200 μl TBS buffer containing as follows [50 mM Tris pH 7.4, 125 mM NaCl, 0.1% TX-100, 1 mM DTT (Sigma) and PICK1 0.5 μM]. The compounds to be tested are added in DMSO to a final concentration of 200 μM (2% DMSO final concentrtaion). The last 11 amino acids of the C-terminus of the GLT1b glutamate transporter (60 μM final) and pure DMSO (2%) can be used as a positive and negative control, respectively.

The samples are incubated at 4° C. under slow rotation for 30 min. The beads are centrifuged at 4000 g for 5 min and subsequently washed in TBS buffer and recentrifuged. The beads containing bound protein are eluted in loading buffer and analyzed by 12% SDS-PAGE and proteins are stained with Gelcode blue stain reagent (Pierce). Active compounds i.e. a blocked pull-down are recognized by a reduced PICK1 band on the SDS-PAGE gel compared to the control pull-down.

### Fluorescence Resonance Energy Transfer (FRET)

To determine whether a compound is able to block binding of a PD ligand, such as the c-terminus of the AMPA receptor GluR2 subunit, to the PICK1 PDZ domain in a cellular system a Fluorescent Resonance Energy Transfer (FRET) assay can be employed. PICK1 is fused to eYFP (eYFP-PICK1) and the C-terminal 29 residues of the AMPA receptor subunit GluR2 is fused to eCFP (eCFP-GluR2 C29). As a control for specificity of the FRET signal an alanine is added to the C-terminal 29 residues of GluR2 (eCFP-GluR2 C29+A) to disrupt the PDZ interaction. Coexpression of eYFP-PICK+eCFP-GluR2 C29 (but not eCFP-GluR2 C29+A) will provide a FRET signal that are reduced by a small molecule compound if it can pass the plasma membrane and bind to the PICK1 PDZ domain.

Fluorescence resonance energy transfer (FRET; see Schmid and Sitte, 2003) is measured with an epi-fluorescence microscope (Carl Zeiss TM210, Germany) using the “three-filter method” according to Xia and Liu (2001). COS7 cells (3×10⁵/well) are seeded on to poly-D-lysine-coated glass coverslips (24 mm diameter). The next day, cells are transiently transfected, using the calcium phosphate precipitation method. Briefly, 1-3 μg cDNA was mixed with CaCl₂ and HBS buffer (280 mM NaCl/10 mM KCl/1.5 mM Na₂HPO₄ is 12 mM dextrose/50 mM HEPES); after 6-10 minutes, the calcium phosphate-DNA precipitate was added to the cells. After 4-5 hours, the cells are washed twice with PBS and briefly treated with glycerol, followed by the addition of FCS-containing medium.

Media are replaced by Krebs-HBS buffer (10 mM HEPES/120 mM NaCl/3 mM KCl/2 mM CaCl₂/2 mM MgCl₂), and images are taken using a 63× oil objective and a LUDL filter wheel that allows for rapid exchange of filters (less than 100 ms). The system is equipped with the following fluorescence filters: CFP filter (I_{CFP}; exc.: 436 nm, dichr.: 455 nm, em.: 480 nm), YFP filter (I_{YFP}; exc.: 500 nm, dichr.: 515 nm, em.: 535 nm) and FRET filter (I_{FRET}: excitation=436 nm, dichroic mirror=455 nm, emission=535 nm). The acquisition of the images is done with MetaMorph (Meta Imaging, Universal Imaging Corporation, V. 4.6.). Background fluorescence is subtracted from all images and fluorescence intensity was measured in cytosolic regions in all images. To calculate a normalized FRET signal (nFRET), the following equation is used: Error! Objects cannot be created from editing field codes., where a and b represents the bleed-through values for YFP and CFP.

### Test Results

### Example 1

### Fluorescence Polarization

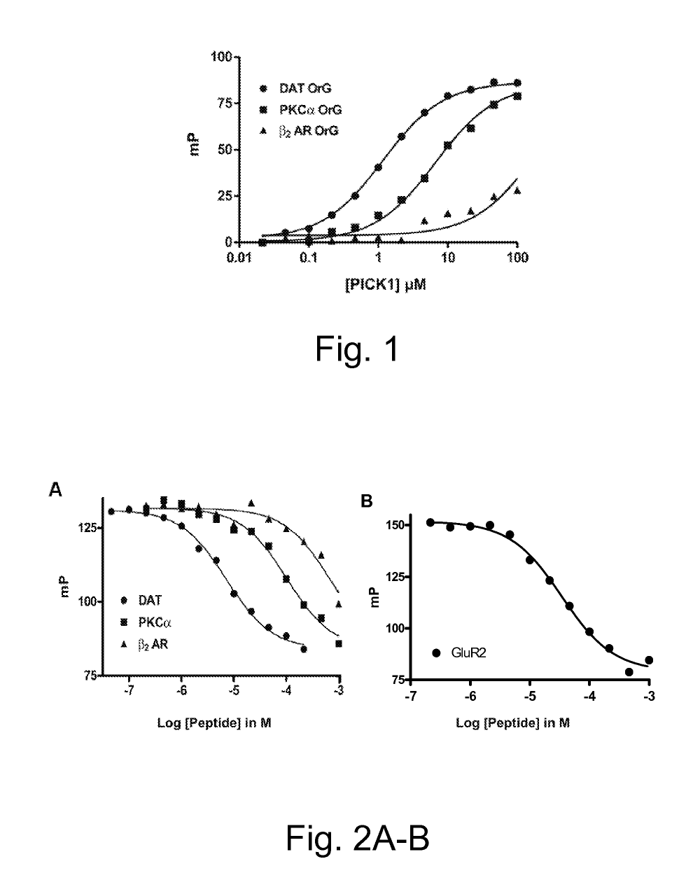

To perform the initial large scale screen of compounds we used a fluorescence polarization (FP) assay as described in Methods. The assay is based on the capability of purified PICK1 to bind a fluorescently conjugated peptide in its PDZ domain binding pocket. In the assay we used peptides corresponding to the 13 C-terminal residues of PKCα, which has a type I PDZ binding sequence -QSAV, and of the human dopamine transporter (hDAT), which has a type II PDZ binding sequence -WLKV, both of which are known to bind PICK1. A peptide corresponding to the 13 C-terminal residues of the β₂ AR was included as a control for the specificity of the saturation binding assay. Like the PKCα sequence, the β₂ AR sequence contains a type I PDZ binding sequence (-DSLL), but unlike the PKCα sequence it was believed not to bind PICK1. The 13-mer peptides used for saturation binding experiments all had an N-terminal cysteine that allowed fluorescent labeling with the sulfhydryl-reactive fluorophore Oregon Green maleimide. In the binding assay we took advantage of the predicted decrease in rotational diffusion of the fluorescently labeled peptides upon binding to a larger protein. Thus, we could detect the decrease in rotational diffusion upon binding of the peptides to PICK1 as an increase in FP. The increase in FP is illustrated by the saturation binding experiments shown in FIG. 1 in which a fixed concentration of fluorescently labeled peptide was titrated with an increasing amount of PICK1 (FIG. 1). The saturation binding experiments suggested that the DAT peptide bound with highest affinity to the purified preparation of PICK1 with an apparent K_{d} value of ˜1 μM. The PKCα peptide bound with an almost 10-fold lower apparent affinity than the DAT peptide, whereas the β₂ AR peptide bound with very low apparent affinity (FIG. 1).

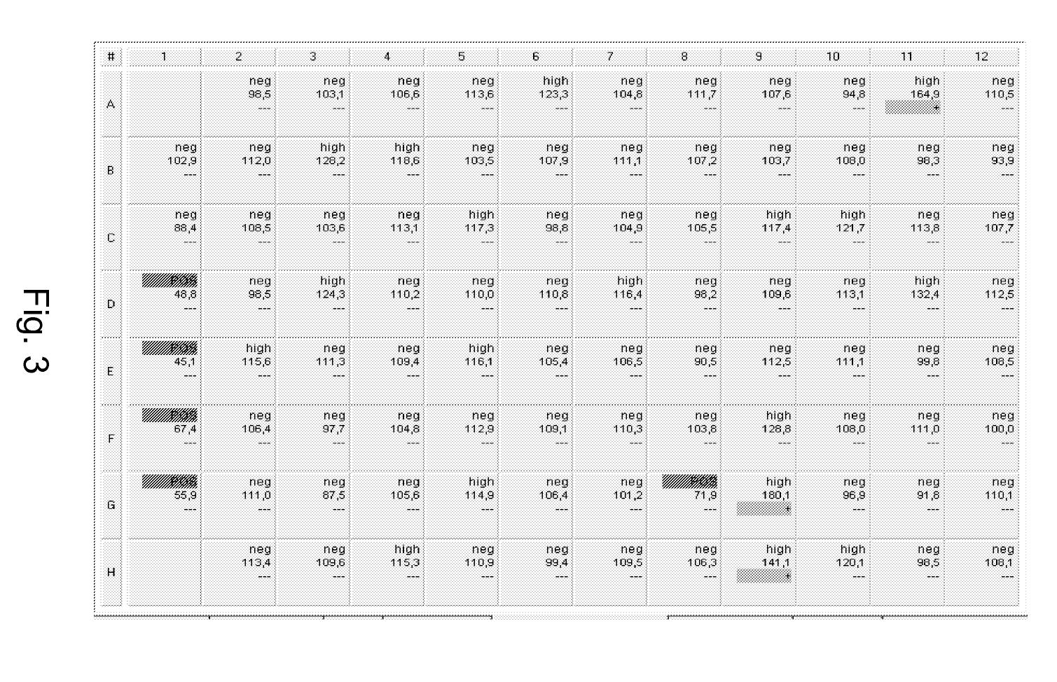

Next, we carried out competition binding experiments in which fixed concentrations of PICK1 and of the fluorescently labeled peptide were titrated with an increasing amount of non-labeled peptide (FIG. 2A). In agreement with the saturation binding experiments, the unlabeled DAT peptide was more than one order of magnitude more potent in displacing the fluorescently tagged tracer than was the unlabeled PKCα peptide. In further agreement with the saturation binding experiments, the β₂ AR peptide was much less potent than the two other peptides. From the competition binding experiments it was possible to calculate Kᵢ values for the interaction of the peptides with PICK1. We should note that the calculated Kᵢ values represent the most accurate estimate of the actual affinities. Thus, the absolute affinities obtained in the saturation binding assay might be affected both by the attached fluorophore and by the ratio between functional and non-functional protein in different purified preparations.

To further verify the specificity of the assay the unlabeled C13 terminal residues of the GluR2 AMPA receptor subunit, which is expected to bind PICK in a similar manner as DAT, was titrated against a fixed concentration of DAT-ORG and PICK, showing the expected sigmoid curve for a one-site competition (FIG. 2B).

Binding of a small-molecule compound to the PDZ binding groove can be detected by its ability to displace the fluorescently labeled peptide resulting in a decreased in FP just as the unlabelled GluR2 peptide.

Using the FP assay we screened ˜40,000 compounds using a setup in which 88 compounds were being tested in one reading session for the capability to block the interaction between PICK1 and the Oregon Green labeled DAT peptide. We used a concentration of 100 μM (10% DMSO final) of each compound. Positive or active wells were recognized by comparing to wells containing DMSO (negative control/mP top level) and wells containing no PICK1 (basal mP level). Active wells were defined as wells giving an mP value below 80% of the negative controls. (FIG. 3)

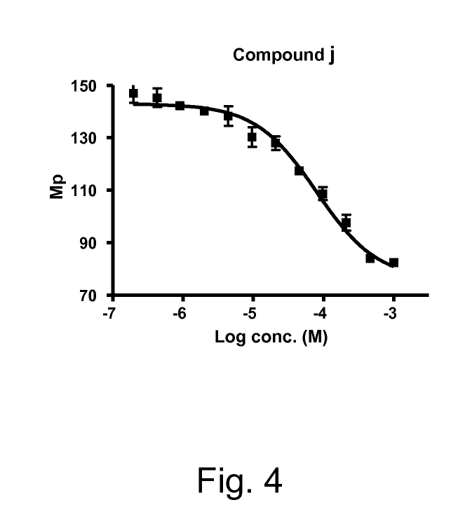

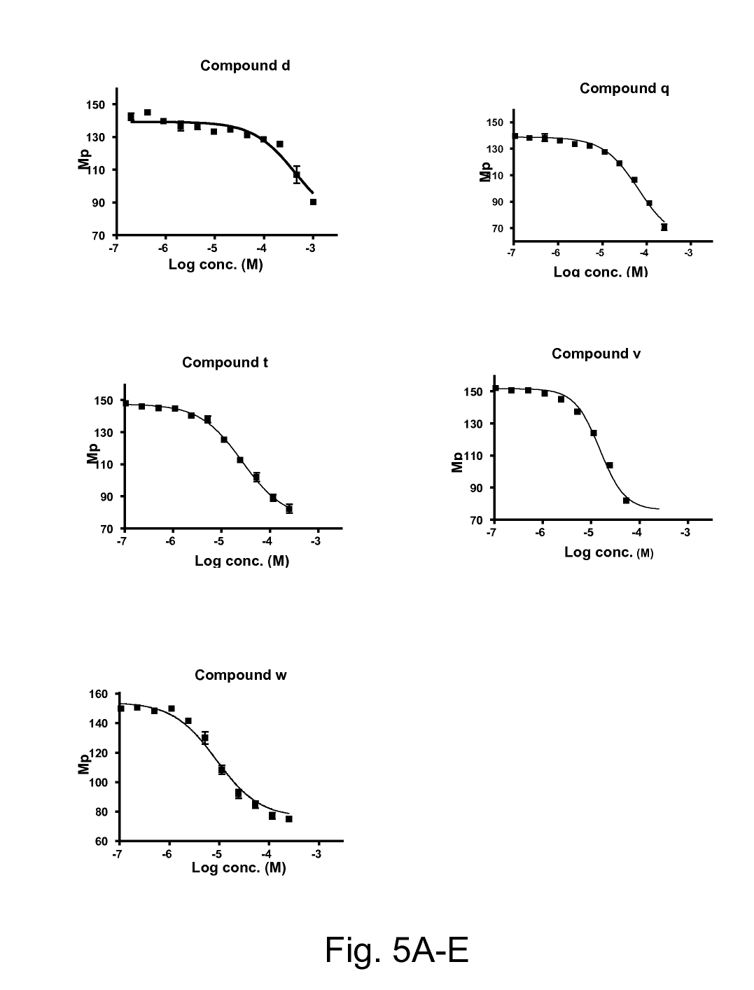

The screening strategy led to identification of a number of active wells. To confirm these active wells, a competition curve, similar to the above with GluR2 peptide, was conducted and the Kᵢ values were determined (FIGS. 4 and 5) and Table 1.

TABLE 1
Calculated Kᵢ values for all the six compounds issued in this patent.

| Compound | Kᵢ (μM) [SE interval] |
|---|---|
| j | 40 [39.3-41.7] |
| d | 67 [58.2-76.1] |
| w | 4.4 [3.86-5.12] |
| t | 13.7 [11.9-15.9] |
| v | 7.7 [7.52-7.84] |
| q | 19.1 [17.6-20.7] |

Kᵢ values were calculated based on the FP assay experiments as described in Methods. Data are means of three independent experiments. The IC₅₀ values used in the estimation of the Kᵢ values were calculated from the means of pIC₅₀ values and the SE interval from the pIC₅₀ ± SE.

### Example 2

### Pull Down Assay

To confirm the specific activity of the compounds found in the FP screen a pull down assay was developed.

In this assay the compounds are tested for the capability to block the interaction between PICK1 and a DAT-peptide fused to GST. Most importantly this assay is not influenced be fluorescent properties of the small molecules.

PICK1 and the GST-DAT fusion protein bound to glutathione-coated sepharose beads were allowed to interact in a buffer containing the compound to be tested. After 30 min. of rotation at 4° C., the beads with bound GST-DAT and possibly bound PICK were peileted and washed before elution in loading buffer and analysis by SDS-PAGE. Proteins were visualized using Gelcode blue stain.

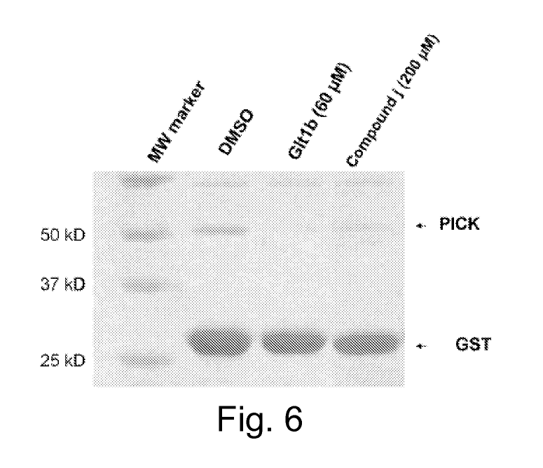

As shown in FIG. 6, GST-DAT efficiently pulled down PICK1 and, as expected, the pull-down was blocked by a peptide corresponding to the C-terminus (13 a.a.) of the GIT1b glutamate transporter. Moreover, we observed that the pull-down of PICK1 by GST-DAT was inhibited by compound (j) as reflected in the weaker PICK1 band and consistent with inhibition of the PICK1 GST-DAT interaction.

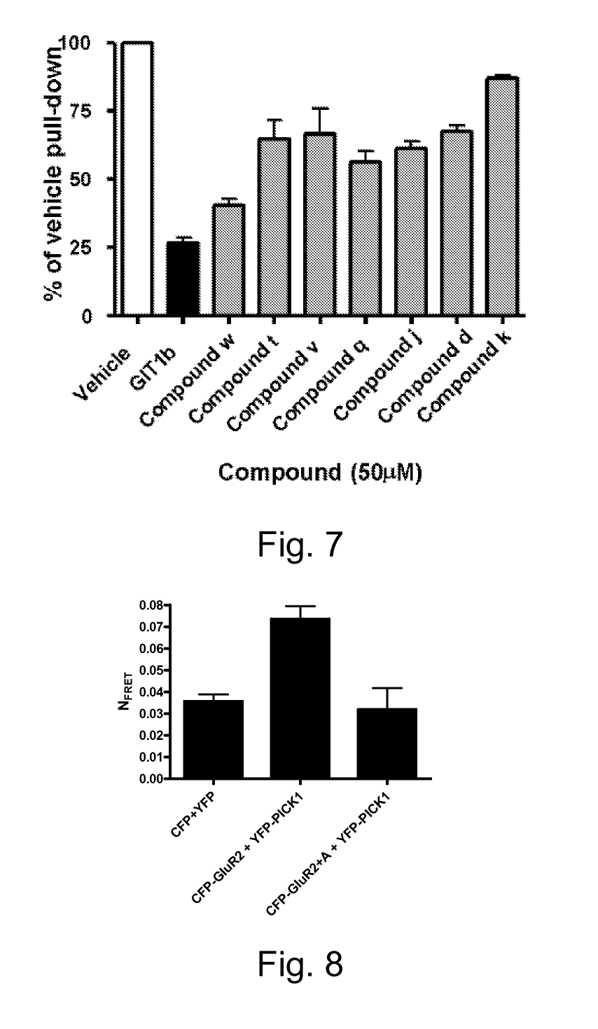

Additional compounds were tested in the same assay using the same concentration of each compound (50 μM) and the results were analyzed by densitometry of the gels. The results are summarized in FIG. 7.

### Example 3

### Disruption of the PICK1 PDZ Interaction in Intact Cellular System

A functional FRET pair dependent on PDZ mediated interaction of PICK1 with GluR2 was established by fusing PICK1 to eYFP (eYFP-PICK1) and the C-terminal 29 residues of GluR2 to eCFP (eCFP-GluR2 C29) as described in Methods. When transiently transfected into COS-7 cells significant FRET was observed from eCFP-GluR2 C29 to eYFP-PICK1, suggesting an interaction between the two proteins (FIG. 8). By introducing an alanine at the C-terminal of GluR2 (eCFP-GluR2 C29+A), which is believed to disrupt the PDZ interaction, the FRET value was reduced to value of the negative control (eCFP+eYFP expressed independently and predicted not to interact). We also tested a fusion protein between eCFP and eYFP (eCFP-eYFP) as a positive control providing a FRET value of 0.361±0.008. Altogether, the data suggest that we are able to monitor the PICK1 GluR2 interaction by FRET in intact living cells (FIG. 8).

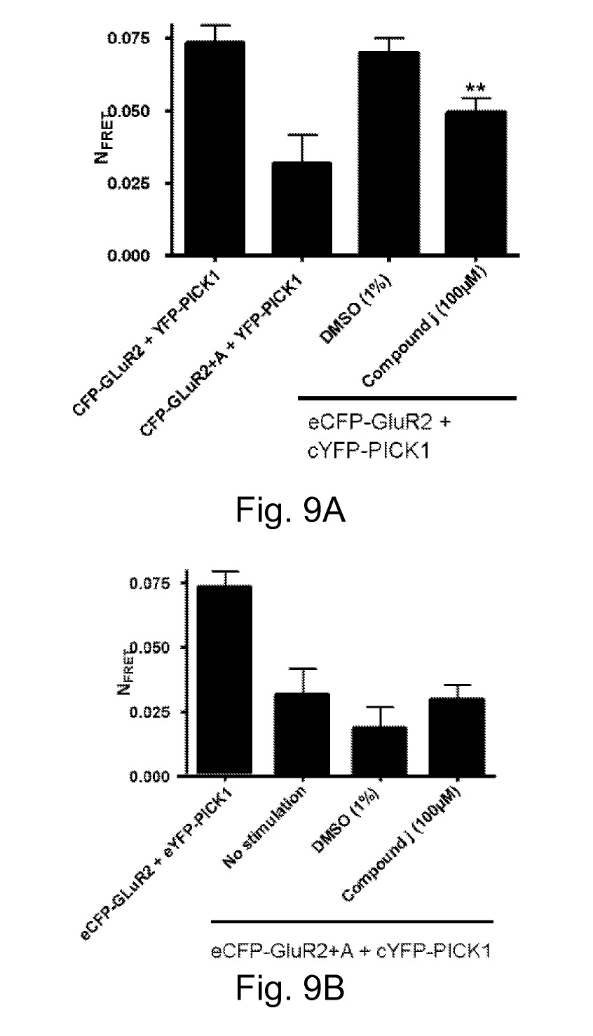

In order to test compounds identified in our screen, cells expressing eYFP-PICK1 together with eCFP-GluR2 C29 and eCFP-GluR2 C29+A were incubated with compound (j) for 20 min prior to image acquisition. Incubation with the compound significantly reduced FRET between eYFP-PICK1 and eCFP-GluR2 C29 (FIG. 9A). No effect was observed of the vehicle (1% DMSO) (FIG. 9A) and no effect was seen for FRET between eYFP-PICK1 and eCFP-GluR2 C29+A (FIG. 9B).

1-24. (canceled)

25. A method for treatment, prevention or alleviation of a disease or a disorder or a condition of a living animal body, including a human, which disorder, disease or condition is responsive to modulation of a PDZ domain, which method comprises the step of administering to such a living animal body in need thereof a therapeutically effective amount of a compound of Formula 1a, 1b, 1c, 1d, 1e or 1f: 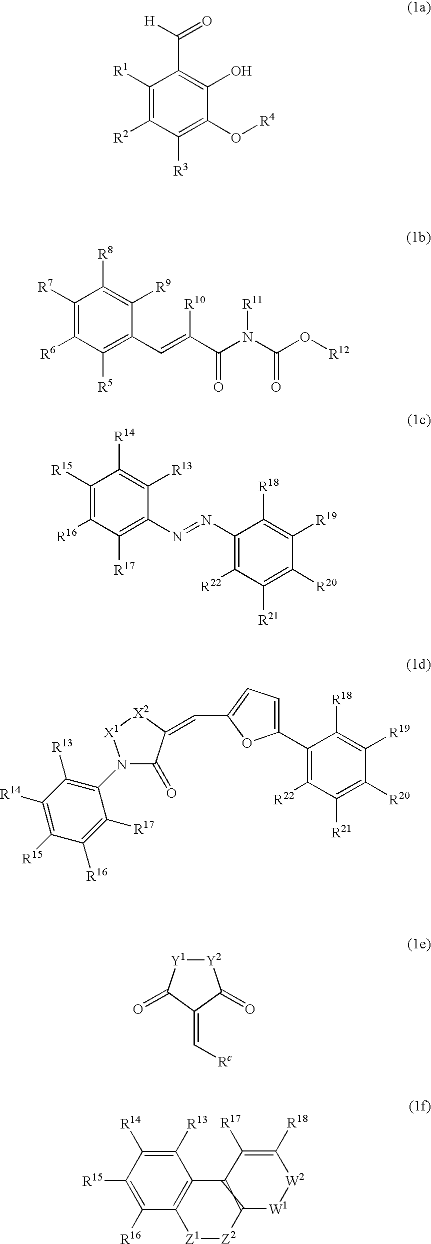
any of its stereoisomers or any mixture of its stereoisomers,
or a pharmaceutically acceptable salt thereof;
for the manufacture of a pharmaceutical composition for the treatment, prevention or alleviation of a disease or a disorder or a condition of a mammal, including a human, which disease or disorder or condition is responsive to modulation of a PDZ domain;
where in Formula 1a, 1b, 1c, 1d, 1e and 1f:
R¹, R² and R³ are independently selected from the group consisting of:
hydrogen, halo, trifluoromethyl, trifluoromethoxy, cyano, nitro, alkyl, hydroxy, alkoxy, formyl, alkylcarbonyl and —(C≡C)ₙ—R^{a}; wherein
n is 0 or 1; and
R^{a} represents an aryl or a heteroaryl group;
which aryl or heteroaryl group is optionally substituted with one or more substituents independently selected from the group consisting of:
halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
wherein R′ and R″ independent of each other are hydrogen or alkyl;
R⁴ represents hydrogen or alkyl;
R⁵, R⁶, R⁷ and R⁸ are independently selected from the group consisting of:
hydrogen, halo, trifluoromethyl, trifluoromethoxy, cyano, nitro, alkyl, hydroxy, alkoxy, formyl, alkylcarbonyl or an aryl or a heteroaryl group;
which aryl or heteroaryl group is optionally substituted with one or more substituents independently selected from the group consisting of:
halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
wherein R′ and R″ independent of each other are hydrogen or alkyl;
R⁹ and R¹⁰ together form —(O—(C═O))—, —O— or —S—; or
R⁹ represents hydrogen or alkyl; and R¹⁰ represents hydrogen, cyano or alkyl;
R¹¹ and R¹² together form —(CHR′—CH₂)—;
wherein R′ represents hydrogen, alkyl or phenyl; or
R¹¹ represents hydrogen or alkyl; and
R¹² represents hydrogen, alkyl, alkenyl or alkynyl;
which alkyl, alkenyl or alkynyl is optionally substituted with an aryl or heteroaryl group;
which aryl or heteroaryl group is optionally substituted with one or more substituents independently selected from the group consisting of:
halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
wherein R′ and R″ independent of each other are hydrogen or alkyl;
R¹³, R¹⁴, R¹⁵, R¹⁶, R¹⁷, R¹⁸, R¹⁹, R²⁰, R²¹ and R²² are independently selected from the group consisting of:
hydrogen, halo, trifluoromethyl, trifluoromethoxy, cyano, nitro, alkyl, hydroxy, alkoxy, formyl, alkylcarbonyl, hydroxycarbonyl, alkoxycarbonyl, R^{b} and —C═N—R^{b}; wherein
R^{b} represents an aryl or a heteroaryl group;
which aryl or heteroaryl group is optionally substituted with one or more substituents independently selected from the group consisting of:
halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
wherein R′ and R″ independent of each other are hydrogen or alkyl;
-X¹-X²- represents —N═(C—R′)— or —NR″—(C═O)—; wherein
wherein R′ and R″ independent of each other are hydrogen or alkyl;
Y¹—Y²— represents 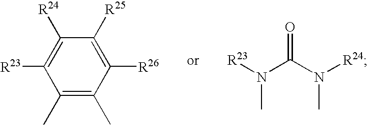
wherein
R²³, R²⁴, R²⁵ and R²⁶ are independently selected from the group consisting of:
hydrogen, halo, trifluoromethyl, trifluoromethoxy, cyano, nitro, alkyl, hydroxy, alkoxy, formyl, alkylcarbonyl, and R^{d};
R^{c} and R^{d} independent of each other represents an aryl or a heteroaryl group;
which aryl or heteroaryl group is optionally substituted with one or more substituents independently selected from the group consisting of:
halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, R^{e}-alkoxy, —NR′R″, —(C═O)NR′R″ or —NR′(C═O)R″;
wherein R′ and R″ independent of each other are hydrogen or alkyl;
R^{e} represents an aryl group;
which aryl group is optionally substituted with one or more substituents independently selected from the group consisting of:
halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy,
—NR′″R″″, —(C═O)NR′″R″″ or —NR′″(C═O)R′″;
wherein R′″ and R″″ independent of each other are hydrogen or alkyl;
-Z¹-Z²- represents —NR′—C(COOR″)— or —(C═O)—(C═O)—; wherein
wherein R′ and R″ independent of each other are hydrogen or alkyl;
—W¹—W²— represents —C(R²⁷R²⁸)— or —CR²⁷═CR²⁸—; wherein
wherein R²⁷ and R²⁸ are independently selected from the group consisting of:
hydrogen, halo, trifluoromethyl, trifluoromethoxy, cyano, nitro, alkyl, hydroxy and alkoxy;
the bond [symbol] represents a single or a double bond.

26. The method according to claim 25, wherein the compound is a compound of Formula 1a 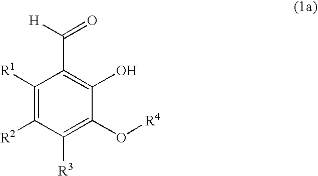
any of its stereoisomers or any mixture of its stereoisomers, or a pharmaceutically acceptable salt thereof; wherein R¹, R², R³ and R⁴ are as defined in claim 25.

27. The method according to claim 25, wherein the compound is a compound of Formula 1b 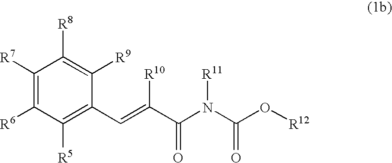
any of its stereoisomers or any mixture of its stereoisomers, or a pharmaceutically acceptable salt thereof; wherein R⁵, R⁶, R⁷, R⁸, R⁹, R¹⁰, R¹¹ and R¹² are as defined in claim 25.

28. The method according to claim 25, wherein the compound is a compound of Formula 1c 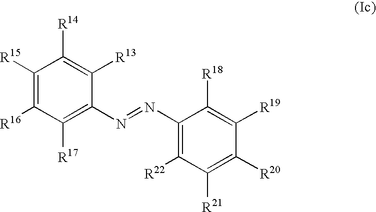
any of its stereoisomers or any mixture of its stereoisomers, or a pharmaceutically acceptable salt thereof; wherein R¹³, R¹⁴, R¹⁵, R¹⁶, R¹⁷, R¹⁸, R¹⁹, R²⁰, R²¹ and R²² are as defined in claim 25.

29. The method according to claim 25, wherein the compound is a compound of Formula 1d 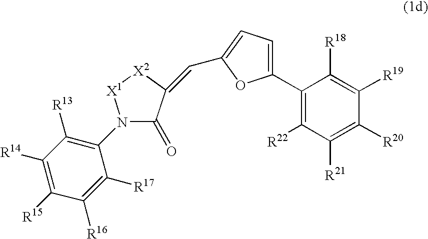
any of its stereoisomers or any mixture of its stereoisomers, or a pharmaceutically acceptable salt thereof; wherein R¹³, R¹⁴, R¹⁵, R¹⁶, R¹⁷, R¹⁸, R¹⁹, R²⁰, R²¹, R²² and -X¹-X²- are as defined in claim 25.

30. The method according to claim 25, wherein the compound is a compound of Formula 1e 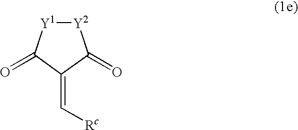
any of its stereoisomers or any mixture of its stereoisomers, or a pharmaceutically acceptable salt thereof; wherein R^{c} and —Y¹—Y²— are as defined in claim 25.

31. The method according to claim 25, wherein the compound is a compound of Formula 1f 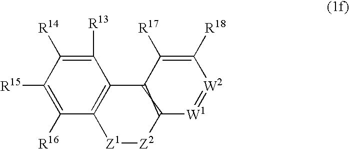
any of its stereoisomers or any mixture of its stereoisomers, or a pharmaceutically acceptable salt thereof; wherein R¹³, R¹⁴, R¹⁵, R¹⁶, R¹⁷, R¹⁸, -Z¹-Z²- and —W¹—W²— and are as defined in claim 25.

32. The method according to claim 25, wherein the compound of Formula 1a-1f is
2,3-Dibromo-5-ethoxy-6-hydroxy-benzaldehyde (a);
3-Ethoxy-2-hydroxy-benzaldehyde (b);
5-Chloro-3-ethoxy-2-hydroxy-benzaldehyde (c);
2,3-Dichloro-5-ethoxy-6-hydroxy-benzaldehyde (d);
5-Bromo-3-ethoxy-2-hydroxy-benzaldehyde (e);
6-Bromo-3-ethoxy-2-hydroxy-benzaldehyde (f);
2-Bromo-3-chloro-5-ethoxy-6-hydroxy-benzaldehyde (g);
3-Ethoxy-2-hydroxy-5-nitro-benzaldehyde (h);
((Z)-2-Cyano-3-phenyl-acryloyl)-carbamic acid ethyl ester (i);
[(Z)-2-Cyano-3-(3,4-dichlorophenyl)-acryloyl]-carbamic acid ethyl ester (j);
3-[(E)-(3-Phenyl-acryloyl)]-oxazolidin-2-one (k);
(Benzo[b]thiophene-2-carbonyl)-carbamic acid prop-2-ynyl ester (l);
4-Phenyl-3-[(E)-3-phenyl-acryloyl)]-oxazolidin-2-one (m);
(6-Bromo-2-oxo-2H-chromene-3-carbonyl)-carbamic acid ethyl ester (n);
(6,8-Dichloro-2-oxo-2H-chromene-3-carbonyl)-carbamic acid butyl ester (o);
(6,8-Diiodo-2-oxo-2H-chromene-3-carbonyl)-carbamic acid ethyl ester (p);
4-tert-Butyl-2-{[1-[5-(4-chloro-phen ylazo)-2-hydroxy-phenyl]-meth-(E)-ylidene]-amino}-phenol (q);
5-(4-Bromo-phenylazo)-2-hydroxy-3-methoxy-benzaldehyde (r);
5-{5-[1-(3-Carboxy-phenyl)-3-methyl-5-oxo-1,5-dihydro-pyrazol-(4Z)-ylidene-methyl]-furan-2-yl}-2-chloro-benzoic acid butyl ester (s);
4-{5-[1-(3-Chloro-4-methyl-phenyl)-3,5-dioxo-pyrazolidin-(4Z)-ylidenemethyl]-furan-2-yl}-benzoic acid ethyl ester (t);
1-(2-Chloro-phenyl)-5-[1-furan-2-yl-meth-(E)-ylidene]-pyrimidine-2,4,6-trione (u);
2-(2-Benzyloxy-5-bromo-benzylidene)-indan-1,3-dione (v);
2-Nitro-phenanthrene-9,10-dione (w);
8-Chloro-3a,4,5,9b-tetrahydro-3H-cyclopenta[c]quinoline-4-carboxylic acid (x);
any of its stereoisomers or any mixture of its stereoisomers, or a pharmaceutically acceptable salt thereof.

33. The method according to claim 25, wherein the disease or disorder or condition is responsive to modulation of a PDZ domain is disease or disorder or condition is responsive to modulation of the PDZ domain of PICK1.

34. The method according to claim 25, wherein the disease or disorder or condition is responsive to modulation of a PDZ domain is acute pain, chronic pain, neuropathic pain, intractable pain, migraine, neurological and psychiatric disorders, depression, anxiety, psychosis, schizophrenia, excitatory amino acid-dependent psychosis, cognitive disorders, dementia, senile dementia, AIDS-induced dementia, stress-related psychiatric disorders, stroke, global ischaemic, focal ischaemic, haemorrhagic stroke, cerebral hypoxia, cerebral ischaemia, cerebral infarction, cerebral ischaemia resulting from thromboembolic or haemorrhagic stroke, cardiac infarction, brain trauma, brain oedema, cranial trauma, brain trauma, spinal cord trauma, bone-marrow lesions, hypoglycaemia, anoxia, neuronal damage following hypoglycaemia, hypotonia, hypoxia, perinatal hypoxia, cardiac arrest, acute neurodegenerative diseases or disorders, chronic neurodegenerative diseases or disorders, brain ischaemia, CNS degenerative disorders, Parkinson's disease, Alzheimer's disease, Huntington's disease, idiopathic Parkinson's Disease, drug induced Parkinson's Disease, amyotrophic lateral sclerosis (ALS), post-acute phase cerebral lesions, chronic diseases of the nervous system, cerebral deficits subsequent to cardiac bypass surgery, cerebral deficits subsequent to grafting, perinatal asphyxia, anoxia from drowning, anoxia from pulmonary surgery. anoxia from cerebral trauma, hypoxia induced nerve cell damage, epilepsy, status epilepticus, seizure disorders, cerebral vasospasm, CNS mediated spasms, motility disorders, muscular spasms, urinary incontinence, convulsions, disorders responsive to anticonvulsants, autoimmune diseases, emesis, nausea, obesity, chemical dependencies, chemical addictions, addictions, withdrawal symptoms, drug induced deficits, alcohol induced deficits, drug addiction, ocular damage, retinopathy, retinal neuropathy, tinnitus, and tardive dyskinesia, inflammatory pain, neurogenic pain, fibromyalgia, chronic fatigue syndrome, nociceptive pain, cancer pain, postoperative pain, migraine, tension-type headache, pain during labour and delivery, breakthrough pain, stroke, drug abuse and cocaine abuse.

35. A compound of Formula 1a or 1b: 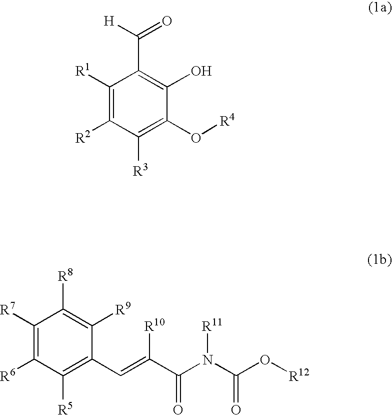
any of its stereoisomers or any mixture of its stereoisomers, or a pharmaceutically acceptable salt thereof; wherein
one of R¹, R² and R³ represents —(C≡C)n-Ra; wherein
wherein n is 0 or 1; and
R^{a} represents an aryl or a heteroaryl group;
which aryl or heteroaryl group is optionally substituted with one or more substituents independently selected from the group consisting of:
halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
wherein R′ and R″ independent of each other are hydrogen or alkyl;
the remaining two of R¹, R² and R³ are independently selected from the group consisting of:
hydrogen, halo, trifluoromethyl, trifluoromethoxy, cyano, nitro, alkyl, hydroxy, alkoxy formyl and alkylcarbonyl;
R⁴ represents hydrogen or alkyl;
R⁵, R⁶, R⁷ and R⁸ are independently selected from the group consisting of:
hydrogen, halo, trifluoromethyl, trifluoromethoxy, cyano, nitro, alkyl, hydroxy, alkoxy or an aryl or a heteroaryl group;
which aryl or heteroaryl group is optionally substituted with one or more substituents independently selected from the group consisting of:
halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
wherein R′ and R″ independent of each other are hydrogen or alkyl;
R⁹ and R¹⁰ together form —(O—(C═O))—, —O— or —S—; or
R⁹ represents hydrogen or alkyl; and R¹⁰ represents hydrogen, cyano or alkyl;
R¹¹ and R¹² together form —(CHR′—CH₂)—;
wherein R′ represents hydrogen, alkyl or phenyl; or
R¹¹ represents hydrogen or alkyl; and
R¹² represents hydrogen, alkyl, alkenyl or alkynyl;
which alkyl, alkenyl or alkynyl is optionally substituted with an aryl or heteroaryl group;
which aryl or heteroaryl group is optionally substituted with one or more substituents independently selected from the group consisting of:
halo, trifluoromethyl, trifluoromethoxy, cyano, nitro, hydroxy, alkoxy, cycloalkoxy, alkoxyalkyl, cycloalkoxyalkyl, formyl, alkylcarbonyl, methylenedioxy, ethylenedioxy, alkyl, cycloalkyl, cycloalkylalkyl, alkenyl, alkynyl, sulfanyl, thioalkoxy, —NR′R″, —(C═O)NR′R″ or —NR′(C═O)R″;
wherein R′ and R″ independent of each other are hydrogen or alkyl.

36. The compound of claim 35, being a compound of Formula 1a 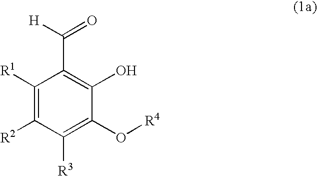
any of its stereoisomers or any mixture of its stereoisomers, or a pharmaceutically acceptable salt thereof; wherein R¹, R², R³ and R⁴ are as defined above.

37. The compound of claim 36, wherein one of R¹, R² and R³ represents —C≡C—R^{a}; any of its stereoisomers or any mixture of its stereoisomers, or a pharmaceutically acceptable salt thereof.

38. The compound of claim 36, wherein one of R¹, R² and R³ represents a monocyclic heteroaryl group; any of its stereoisomers or any mixture of its stereoisomers, or a pharmaceutically acceptable salt thereof.

39. The compound of claim 35, being a compound of Formula 1b 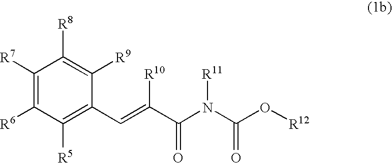
any of its stereoisomers or any mixture of its stereoisomers, or a pharmaceutically acceptable salt thereof; wherein R⁵, R⁶, R⁷, R⁸, R⁹, R¹⁰, R¹¹ and R¹² are as defined above.

40. The compound of claim 39, wherein R¹² represents substituted alkynyl; any of its stereoisomers or any mixture of its stereoisomers, or a pharmaceutically acceptable salt thereof.

41. The compound of claim 39, being a compound of Formula 1b1, 1b2, 1b3 or 1b4: 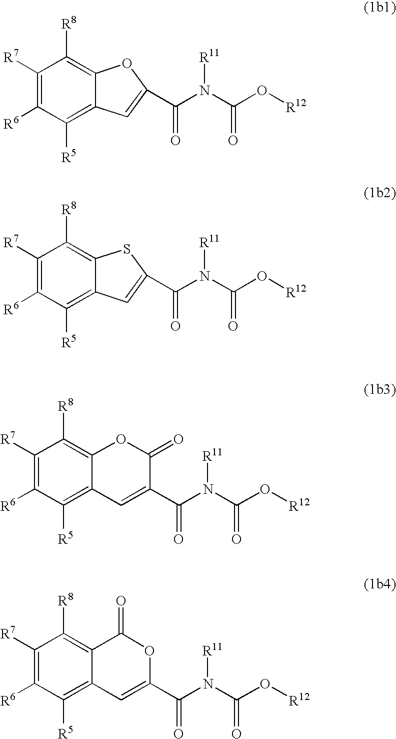
any of its stereoisomers or any mixture of its stereoisomers, or a pharmaceutically acceptable salt thereof; wherein one of R⁵, R⁶, R⁷ and R⁸ is an optionally substituted aryl or a heteroaryl group; and the remaining three of R⁵, R⁶, R⁷ and R⁸ and R¹¹ and R¹² are as defined above.

42. The compound of claim 35, which is
4-Ethoxy-3-hydroxy-biphenyl-2-carbaldehyde;
4-Ethoxy-2′-fluoro-3-hydroxy-biphenyl-2-carbaldehyde;
4-Ethoxy-3′-fluoro-3-hydroxy-biphenyl-2-carbaldehyde;
4-Ethoxy-4′-fluoro-3-hydroxy-biphenyl-2-carbaldehyde;
4-Ethoxy-3-hydroxy-2′-methoxy-biphenyl-2-carbaldehyde;
4-Ethoxy-3-hydroxy-3′-methoxy-biphenyl-2-carbaldehyde;
4-Ethoxy-3-hydroxy-4′-methoxy-biphenyl-2-carbaldehyde;
3-Ethoxy-2-hydroxy-6-pyridin-3-yl-benzaldehyde;
3-Ethoxy-6-furan-2-yl-2-hydroxy-benzaldehyde;
3-Ethoxy-6-furan-3-yl-2-hydroxy-benzaldehyde;
3-Ethoxy-2-hydroxy-6-thiophen-2-yl-benzaldehyde;
3-Ethoxy-2-hydroxy-6-thiophen-3-yl-benzaldehyde;
5-Ethoxy-4-hydroxy-biphenyl-3-carbaldehyde;
3-Ethoxy-5-furan-2-yl-2-hydroxy-benzaldehyde;
3-Ethoxy-5-furan-3-yl-2-hydroxy-benzaldehyde;
3-Ethoxy-2-hydroxy-5-thiophen-2-yl-benzaldehyde;
3-Ethoxy-2-hydroxy-5-thiophen-3-yl-benzaldehyde;
6-Chloro-4-ethoxy-3-hydroxy-biphenyl-2-carbaldehyde;
((Z)-3-Biphenyl-3-yl-2-cyano-acryloyl)-carbamic acid isopropyl ester;
[(Z)-2-Cyano-3-(3-furan-2-yl-phenyl)-acryloyl]-carbamic acid isopropyl ester;
((Z)-3-Biphenyl-4-yl-2-cyano-acryloyl)-carbamic acid ethyl ester;
[(Z)-2-Cyano-3-(4-furan-2-yl-phenyl)-acryloyl]-carbamic acid ethyl ester;
(2-Oxo-6-phenyl-2H-chromene-3-carbonyl)-carbamic acid ethyl ester;
(6-Furan-2-yl-2-oxo-2H-chromene-3-carbonyl)-carbamic acid ethyl ester;
(6-Furan-3-yl-2-oxo-2H-chromene-3-carbonyl)-carbamic acid ethyl ester;
(2-Oxo-6-thiophen-2-yl-2H-chromene-3-carbonyl)-carbamic acid ethyl ester;
(2-Oxo-6-thiophen-3-yl-2H-chromene-3-carbonyl)-carbamic acid ethyl ester;
3-Ethoxy-2-hydroxy-6-phenylethynyl-benzaldehyde;
3-Ethoxy-2-hydroxy-6-pyridin-3-ylethynyl-benzaldehyde;
3-Ethoxy-2-hydroxy-6-(4-methoxy-phenylethynyl)-benzaldehyde;
3-Ethoxy-2-hydroxy-6-thiophen-3-ylethynyl-benzaldehyde;
3-Ethoxy-2-hydroxy-5-phenylethynyl-benzaldehyde;
3-Chloro-5-ethoxy-6-hydroxy-2-phenylethynyl-benzaldehyde;
(Benzo[b]thiophene-2-carbonyl)-carbamic acid phenylethynyl ester;
(Benzo[b]thiophene-2-carbonyl)-carbamic acid furan-2-ylethynyl ester;
(Benzo[b]thiophene-2-carbonyl)-carbamic acid thiophen-2-ylethynyl ester;
(Benzo[b]thiophene-2-carbonyl)-carbamic acid furan-3-ylethynyl ester;
(Benzo[b]thiophene-2-carbonyl)-carbamic acid thiophen-3-ylethynyl ester;
[(Z)-2-Cyano-3-(3,4-dichloro-phenyl)-acryloyl]-carbamic acid methyl ester;
[(Z)-2-Cyano-3-(3,4-dichloro-phenyl)-acryloyl]-carbamic acid isopropyl ester;
[(Z)-2-Cyano-3-(3,4-dichloro-phenyl)-acryloyl]-carbamic acid propyl ester;
((Z)-2-Cyano-3-phenyl-acryloyl)-carbamic acid methyl ester;
((Z)-2-Cyano-3-phenyl-acryloyl)-carbamic acid isopropyl ester;
(Benzo[b]thiophene-2-carbonyl)-carbamic acid ethyl ester;
(Benzo[b]thiophene-2-carbonyl)-carbamic acid vinyl ester;
any of its stereoisomers or any mixture of its stereoisomers, or a pharmaceutically acceptable salt thereof.

43. A pharmaceutical composition, comprising a therapeutically effective amount of a compound of claim 35, any of its stereoisomers or any mixture of its stereoisomers, or a pharmaceutically acceptable salt thereof, together with at least one pharmaceutically acceptable carrier, excipient or diluent.

44. A method for the treatment, prevention or alleviation of a disease or a disorder or a condition of a mammal, including a human, which disease or disorder or condition is responsive to modulation of a PDZ domain, comprising the step of:
administering to said mammal a therapeutically effective amount of said compound of claim 35, any of its stereoisomers or any mixture of its stereoisomers, or a pharmaceutically acceptable salt thereof.
